(19) United States
(12) Patent Application Publication
(10) Pub. No.: US 2021/0163459 A1
(43) Pub. Date: Jun. 3, 2021

(54) PESTICIDALLY ACTIVE AZETIDINE SULFONES AMIDE ISOXAZOLINE DERIVATIVES

(71) Applicant: SYNGENTA CROP PROTECTION AG, Basel (CH)

(72) Inventors: André JEANGUENAT, Stein (CH); Myriem EL QACEMI, Stein (CH); Aurelien BIGOT, Stein (CH)

(73) Assignee: SYNGENTA CROP PROTECTION AG, Basel (CH)

(21) Appl. No.: 17/253,206

(22) PCT Filed: Jun. 17, 2019
(86) PCT No.: PCT/EP2019/065893

§ 371 Date: Dec. 17, 2020

(30) Foreign Application Priority Data
Jun. 19, 2018 (EP) 18178657.5

(51) Int. Cl. C07D 413/12; A01N 43/80; A01N 25/14; A01N 25/04

(52) U.S. Cl. CPC C07D 413/12; A01N 25/04; A01N 25/14; A01N 43/80

(57) ABSTRACT

Compounds of formula (I), wherein the substituents are as defined in claim 1, and the agrochemically acceptable salts, stereoisomers, enantiomers, tautomers and N-oxides of those compounds, can be used as insecticides and can be prepared in a manner known per se.

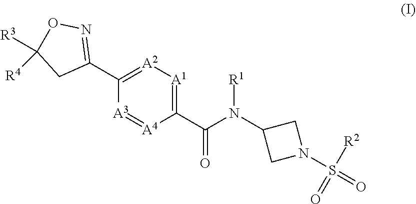

The present invention relates to pesticidally active, in particular insecticidally active, azetidine sulfonamide isoxazolines, to compositions comprising those compounds, and to their use for controlling animal pests (including arthropods and in particular insects or representatives of the order Acarina). Certain isoxazoline derivatives with insecticidal properties are disclosed, for example, in WO2011067272.

There have now been found novel pesticidally active azetidine sulfonamide isoxazoline derivatives.

Accordingly, as an embodiment 1, the present invention relates to compounds of formula (I)

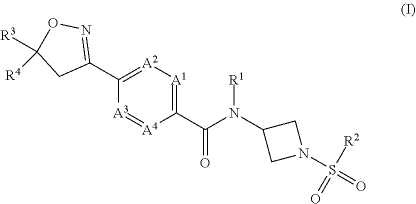

wherein
A¹, A², A³ and A⁴, independently of one another, are C—H, C—R⁵ or nitrogen; wherein not more than two of A¹, A², A³ and A⁴ are N;
R¹ is selected from C₃-C₆cycloalkylcarbonyl, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}) and —R^{1a}C(═O)N(R^{1b})(R^{1c});
R^{1a} is —(CR^{1d}R^{1e})ₙ—;
R^{1b} and R^{1c} are independently selected from H and C₁-C₈alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R² is C₁-C₈alkyl or C₁-C₈haloakyl;
R³ is C₁-C₈haloalkyl;
R⁴ is aryl, aryl substituted by one to three R⁷, heteroaryl or heteroaryl substituted by one to three R⁷;
R⁵ is independently selected from halogen, cyano, nitro, C₁-C₈alkyl, C₃-C₆cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy and C₁-C₈alkoxycarbonyl-, or two R⁵ on adjacent carbon atoms together form a —CH₂—CH₂—CH₂— bridge, a —CH═CH—CH═CH— bridge or a —N═CH—CH═CH— bridge; and
R⁷ is independently selected from halogen, cyano, nitro, C₁-C₈alkyl, C₁-C₈haloalkyl, C₁-C₈alkoxy and C₁-C₈haloalkoxy;
or an agrochemically acceptable salt, stereoisomer, enantiomer, tautomer and N-oxide thereof.

Compounds of formula (I) which have at least one basic centre can form, for example, acid addition salts, for example with inorganic acids such as mineral acids, for example perchloric acid, sulfuric acid, nitric acid, a phosphorus acid or a hydrohalic acid, with organic carboxylic acids, such as C₁-C₄alkanecarboxylic acids which are unsubstituted or substituted, for example by halogen, for example acetic acid or trifluoroacetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

Where substituents are indicated as being substituted, this means that they carry one or more identical or different substituents, e.g. one to three substituents. Where a group is indicated as being substituted, e.g. alkyl, this includes those groups that are part of other groups, e.g. the alkyl in alkoxy or phenyl in phenyloxy. The number of substituents does not exceed the number of available C—H and N—H bonds.

The alkyl groups occurring in the definitions of the substituents can be straight-chain or branched and are, but are not limited to, methyl, ethyl, n-propyl, isopropyl, n-butyl, sec-butyl, iso-butyl, tert-butyl, pentyl, hexyl, nonyl, decyl and their branched isomers. Alkylsulfanyl, alkylsulfinyl, alkylsulfonyl, alkoxy, alkenyl and alkynyl radicals are derived from the alkyl radicals mentioned. The alkenyl and alkynyl groups can be mono- or polyunsaturated.

Halogen is generally fluorine, chlorine, bromine or iodine. This also applies, correspondingly, to halogen in combination with other meanings, such as haloalkyl or halophenyl.

Haloalkyl groups preferably have a chain length of from 1 to 6 carbon atoms. Haloalkyl is, but is not limited to, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl.

Alkoxy is, but is not limited to, methoxy, ethoxy, propoxy, i-propoxy, n-butoxy, isobutoxy, sec-butoxy and tert-butoxy and also the isomeric pentyloxy and hexyloxy radicals.

Alkoxyalkyl is, but are not limited to, methoxymethyl, methoxyethyl, ethoxymethyl, ethoxyethyl, n-propoxymethyl, n-propoxyethyl, isopropoxymethyl or isopropoxyethyl.

Alkoxycarbonyl is, but are not limited to, methoxycarbonyl (which is C₁alkoxycarbonyl), ethoxycarbonyl, propoxycarbonyl, isopropoxycarbonyl, n-butoxycarbonyl, tert-butoxycarbonyl, n-pentoxycarbonyl or hexoxycarbonyl.

The cycloalkyl groups preferably have from 3 to 6 ring carbon atoms and are, for example, cyclopropyl, cyclobutyl, cyclopentyl and cyclohexyl.

As used herein, the term “C₂-C₈alkynyl” refers to a straight or branched hydrocarbon chain radical group consisting solely of carbon and hydrogen atoms, containing at least one triple bond, having from two to eight carbon atoms, and which is attached to the rest of the molecule by a single bond. Examples of C₂-C₈ alkynyl include, but are not limited to, ethynyl, prop-1-ynyl, but-1-ynyl and but-2-ynyl.

As used herein, the term “C₂-C₈alkenyl” refers to a straight or branched hydrocarbon chain radical group consisting solely of carbon and hydrogen atoms, containing at least one double bond, having from two to eight carbon atoms, and which is attached to the rest of the molecule by a single bond. Examples of C₂-C₈alkenyl include, but are not limited to, prop-1-enyl, but-1-enyl and but-2-enyl.

Aryl means a six to fourteen membered aromatic carbocyclic ring system which can be mono-, bi- or tricyclic. Examples of such rings include phenyl, indenyl, naphthalenyl, anthranyl, or phenanthrenyl. A preferred aryl group is phenyl.

Heteroaryl stands for a five to six membered aromatic ring system comprising 1 to 3 hetero atoms selected from the group consisting of nitrogen, oxygen and sulfur, it not being possible for the ring system to contain more than 2 oxygen atoms or more than 2 sulfur atoms. Examples of heteroaryl are furyl, thienyl, pyrrolyl, imidazolyl, pyrazolyl, thiazolyl, isothiazolyl, oxazolyl, isoxazolyl, oxadiazolyl, thiadiazolyl, triazolyl, tetrazolyl, pyridyl, pyridazinyl, pyrimidinyl, pyrazinyl. A preferred heteroaryl group is pyridyl.

The compounds of formula I according to the invention also include hydrates which may be formed during the salt formation.

Embodiment 2: The compounds according to embodiment 1 of formula (I)

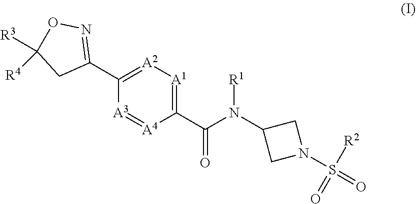

wherein
A¹, A², A³ and A⁴ are independently selected from C—H and C—R⁵;
R¹ is selected from C₃-C₆cycloalkylcarbonyl, —R^{1a}OC(═O)R^{1b}, —R^{1a}OC(═O)OR^{1b}, —R^{1a}N(R^{1c})C(═O)OR^{1b}, —R^{1a}OC(═O)NHR^{1b}, —R^{1a}OC(═O)N(R^{1b})(R^{1c}), —R^{1a}C(═O)NHR^{1b}, —and R^{1a}C(═O)N(R^{1b})(R^{1c});
R^{1a} is —(CR^{1d}R^{1e})ₙ—;
R^{1b} and R^{1c} are independently selected from H and C₁-C₈alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R² is C₁-C₈alkyl or C₁-C₈haloakyl;
R³ is C₁-C₈haloalkyl;
R⁴ is phenyl which is unsubstituted or substituted by one to three R⁷;
R⁵ is independently selected from halogen, cyano, nitro, C₁-C₈alkyl, C₃-C₆cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy and C₁-C₈alkoxycarbonyl-, or two R⁵ on adjacent carbon atoms together form a —CH₂—CH₂—CH₂— bridge, a —CH═CH—CH═CH— bridge or a —N═CH—CH═CH— bridge; and
R⁷ is independently selected from halogen and C₁-C₈haloalkyl;
or an agrochemically acceptable salt, stereoisomer, enantiomer, tautomer and N-oxide thereof.

Embodiment 3: The compound according to embodiment 1 or 2 wherein

A², A³ and A⁴ are C—H and A is C—R⁵;
R⁵ is C₁-C₄alkyl, preferably methyl.

Embodiment 4: The compound according to any one of embodiments 1 to 3 wherein

R⁴ is phenyl which is substituted by one to three R⁷ and wherein R⁷ is halogen or C₁-C₈haloalkyl.

Embodiment 5: The compound according to any one of embodiments 1 to 4 wherein

R⁴ is selected from 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3,5-dichloro-4-fluoro-phenyl and 3,4,5-trichloro-phenyl; more preferably R⁴ is selected from 3-chloro-5-trifluoromethyl-phenyl, 3,5-dichloro-phenyl, 3,5-bis-(trifluoromethyl)-phenyl, 3,5-dichloro-4-fluoro-phenyl and 3,4,5-trichloro-phenyl.

Embodiment 6: The compound according to any one of embodiments 1 to 5 wherein

R³ is —CF₃.

Embodiment 7: The compound according to any one of embodiments 1 to 6 wherein

R² is selected from methyl, ethyl, propyl, isopropyl, CH₂F and CHF₂, preferably R² is selected from methyl, ethyl and CHF₂, more preferably R² is CHF₂.

Embodiment 8: The compound according to any one of embodiments 1 to 7 wherein

R¹ is selected from —R^{1a}OC(═O)OR^{1b} and —R^{1a}N(R^{1c})C(═O)OR^{1b}; wherein R^{1a} is —(CR^{1d}R^{1e})ₙ—;
R^{1b} and R^{1c} are independently selected from C₁-C₈alkyl, wherein each alkyl group is unsubstituted or substituted with one to three halogen atoms or with a cyano group;
R^{1d} and R^{1e} are independently selected from H and C₁-C₄alkyl;
n is 1 or 2.

Embodiment 9: The compound according to any one of embodiments 1 to 8 wherein

R¹ is selected from —CH₂C(═O)OCH₃ and —CH₂N(CH₃)C(═O)OCH₃.

The compounds of the invention may be made by a variety of methods as shown in Schemes 1 to 5.

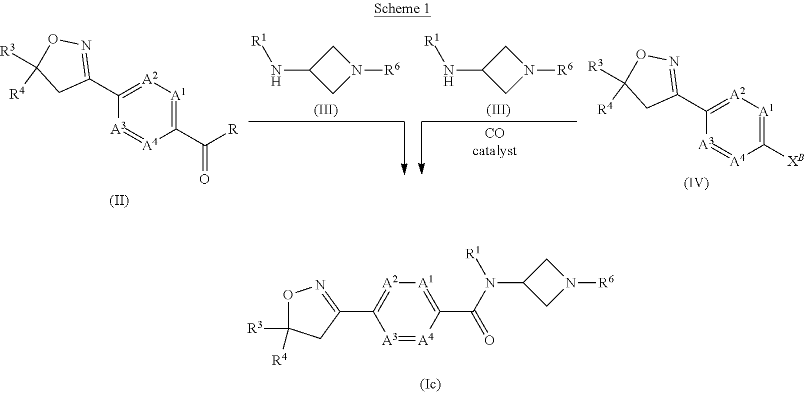

1) Compounds of formula (Ic), wherein R⁶ is S(O)₂R², hydrogen or an amine protecting group (usual removable amine protecting groups are described in “Greene's Protective Groups in Organic Synthesis”, 4th ed., Wuts, P. G. M., Greene, T. W. 2007, J. Wiley, Hoboken, N.J.), can be prepared by reacting a compound of formula (II) wherein R is OH, C₁-C₆alkoxy or Cl, F or Br, with an amine of formula (III), as shown in Scheme 1. When R is OH such reactions are usually carried out in the presence of a coupling reagent, such as N,N′-dicyclohexylcarbodiimide (“DCC”), 1-ethyl-3-(3-dimethylamino-propyl)carbodiimide hydrochloride (“EDC”) or bis(2-oxo-3-oxazolidinyl)phosphonic chloride (“BOP—Cl”), in the presence of a base, and optionally in the presence of a nucleophilic catalyst, such as hydroxybenzotriazole (“HOBT”). When R is Cl, such reactions are usually carried out in the presence of a base, and optionally in the presence of a nucleophilic catalyst. Alternatively, it is possible to conduct the reaction in a biphasic system comprising an organic solvent, preferably ethyl acetate, and an aqueous solvent, preferably a solution of sodium hydrogen carbonate. When R is C₁-C₆alkoxy it is sometimes possible to convert the ester directly to the amide by heating the ester and amine together in a thermal process. Suitable bases include pyridine, triethylamine, 4-(dimethylamino)-pyridine (“DMAP”) or diisopropylethylamine (Hunig's base). Preferred solvents are N,N-dimethylacetamide, tetrahydrofuran, dioxane, 1,2-dimethoxyethane, ethyl acetate and toluene. The reaction is carried out at a temperature of from 0° C. to 100° C., preferably from 15° C. to 30° C., in particular at ambient temperature. Amines of formula (III) are either known in the literature or can be prepared using methods known to a person skilled in the art.

2) Acid halides of formula (II), wherein R is Cl, F or Br, may be made from carboxylic acids of formula (II), wherein R is OH, under standard conditions, as described for example in WO 2009/080250.

3) Carboxylic acids of formula (II), wherein R is OH, may be formed from esters of formula (II), wherein R is C₁-C₆alkoxy as described for example in WO 2009/080250.

4) Compounds of formula (Ic) can be prepared by reacting a compound of formula (IV) wherein X^{B} is a leaving group, for example a halogen, such as bromo, with carbon monoxide and an amine of formula (III), in the presence of a catalyst, such as palladium(II) acetate or bis(triphenylphosphine)palladium(II) dichloride, optionally in the presence of a ligand, such as triphenylphosphine, and a base, such as sodium carbonate, pyridine, triethylamine, 4-(dimethylamino)-pyridine (“DMAP”) or diisopropylethylamine (Hunig's base), in a solvent, such as water, N,N-dimethylformamide or tetrahydrofuran. The reaction is carried out at a temperature of from 50° C. to 200° C., preferably from 100° C. to 150° C. The reaction is carried out at a pressure of from 50 to 200 bar, preferably from 100 to 150 bar.

5) Compounds of formula (IV) wherein X^{B} is a leaving group, for example a halogen, such as bromo, can be made by a various of methods, for example as described in WO 2009/080250.

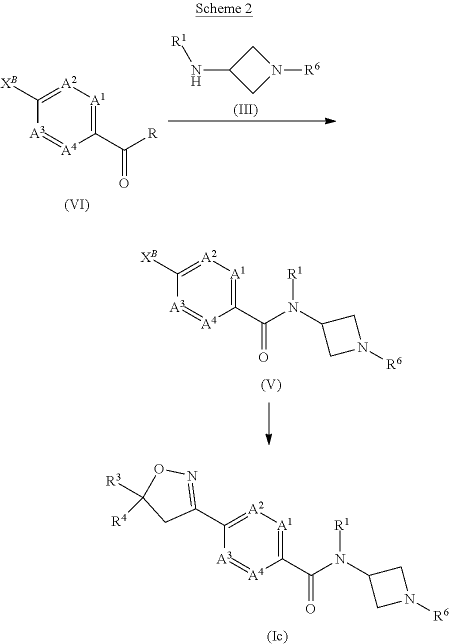

6) Alternatively, compounds of formula (Ic), wherein R⁶ is S(O)₂R², hydrogen or an amine protecting group (usual removable amine protecting groups are described in “Greene's Protective Groups in Organic Synthesis”, 4th ed., Wuts, P. G. M., Greene, T. W. 2007, J. Wiley, Hoboken, N.J.), can be prepared by various methods from an intermediate of formula (V) as shown in Scheme 2 wherein X^{B} is a leaving group, for example a halogen, such as bromo, or X^{B} is cyano, formyl or acetyl according to similar methods to those described in WO 2009/080250. An intermediate of formula (V) can be prepared for example from an intermediate of formula (VI) as described in the same reference.

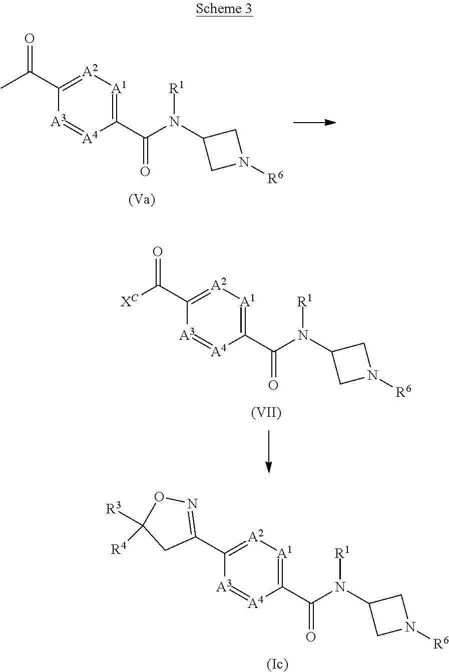

7) Alternatively, compounds of formula (Ic) can be prepared by various methods from an intermediate of formula (VII) as shown in Scheme 3 wherein X^{C} is CH═C(R³)R⁴, or CH₂C(OH)(R³)R wherein R³ and R⁴ are as defined for a compound of formula (I) according to similar methods to those described in WO 2009/080250.

8) Compounds of formula (VII) wherein X^{C} is CH═C(R³)R⁴, or CH₂C(OH)(R³)R⁴ can be prepared from a compound of formula (Va) or from a compound of formula (VII) wherein X^{C} is CH₂-halogen using similar methods to those described in WO 2009/080250.

9) Compounds of formula (VII) wherein X^{C} is CH₂-halogen, such as bromo or chloro, can be prepared by reacting a methyl ketone of formula (Va), with a halogenating agent, such as bromine or chlorine, in a solvent, such as acetic acid, at a temperature of from 0° C. to 50° C., preferably from ambient temperature to 40° C.

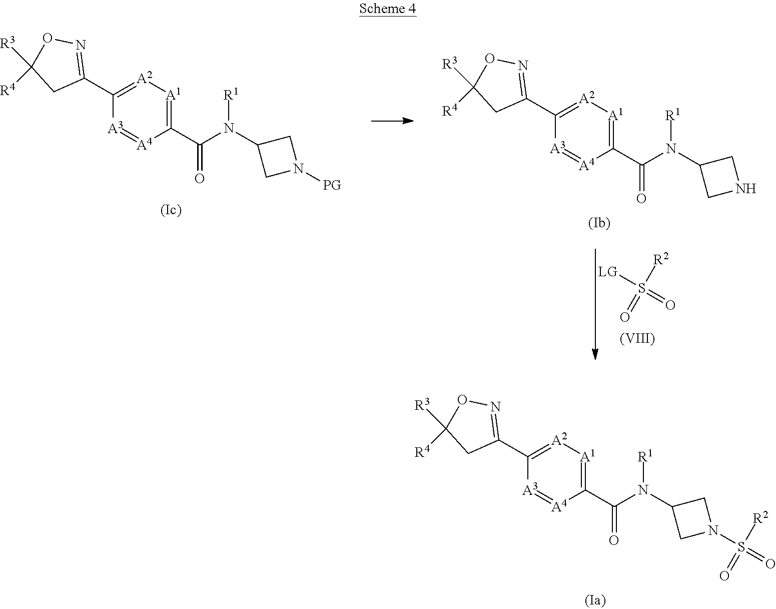

10) Compounds of formula (Ia) can be prepared by reacting a compound of formula (Ib) with a sulfonyl compound of formula (VIII), wherein LG is a suitable leaving group, such as a halogen atom, such as chlorine, or an imidazole or a substituted phenoxy group, as shown in Scheme 4. Such reactions are usually carried out in the absence or in the presence of a base, such as sodium hydroxide, trimethylamine, sodium hydrogenocarbonate, sodium carbonate, using methods known to a person skilled in the art.

11) In addition, compounds of formula (Ib) can be prepared by deprotecting the amine in a compound of formula (Ic), where PG is an amine protecting group (removable protecting groups are described in “Greene's Protective Groups in Organic Synthesis”, 4th ed., Wuts, P. G. M., Greene, T. W. 2007, J. Wiley, Hoboken, N.J.) as shown in Scheme 4. Such reactions are known to a person skilled in the art. The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N, N-diethylaniline, may also act as solvents or diluents.

The reaction is advantageously carried out in a temperature range from approximately −80° C. to approximately +140° C., preferably from approximately −30° C. to approximately +100° C., in many cases in the range between ambient temperature and approximately +80° C.

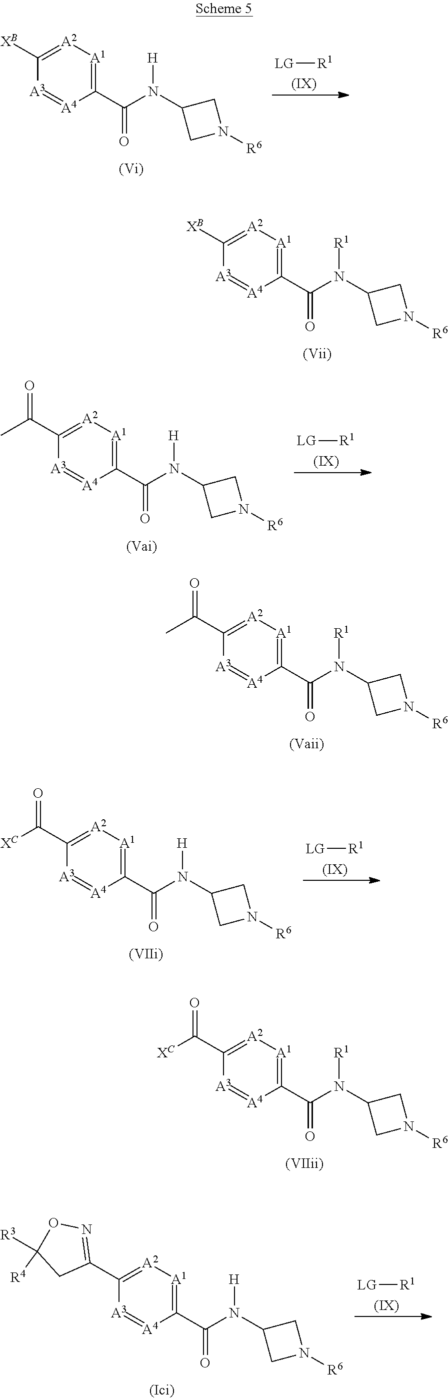

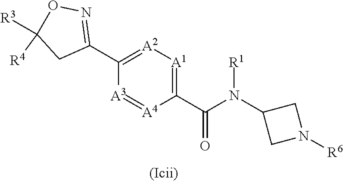

12) Alternatively, compounds of formula (Vii), wherein R⁶ is S(O)₂R², hydrogen or an amine protecting group (usual removable amine protecting groups, such as a BOC protecting group, are described in “Greene's Protective Groups in Organic Synthesis”, 4th ed., Wuts, P. G. M., Greene, T. W. 2007, J. Wiley, Hoboken, N.J.), can be prepared by various methods from an intermediate of formula (Vi) as shown in Scheme 5 wherein X^{B} is a leaving group, for example a halogen, such as bromo, or X^{B} is cyano, formyl or acetyl using usual conditions for an alkylation of acylation, as known from a person skilled in the art, using a compound of formula (IX), wherein LG is a suitable leaving group, such as a halogen atom, such as chlorine, or an imidazole or a substituted phenoxy group. Similarly, compounds of formula (Vaii), (VIIii) and (Icii) could be prepared starting from compounds of formula (Vai), (VIIi) and (Ici) respectively.

A compound of formula (I) can be converted in a manner known per se into another compound of formula (I) by replacing one or more substituents of the starting compound of formula (I) in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

The present invention also provides intermediates useful for the preparation of compounds of formula (I). Certain intermediates are novel and as such form a further aspect of the invention. One group of novel intermediates are compounds of formula (Int-1)

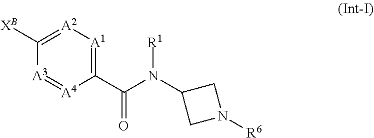

wherein A¹, A², A³, A⁴ and R¹ are as defined for a compound according to any one of embodiments 1 to 9 and R⁶ is hydrogen or S(O)₂R² and X^{B} is a halogen, such as bromo, or X^{B} is cyano, formyl, CH═N—OH or acetyl; and a salt or N-oxide thereof. The preferences for A¹, A², A³, A⁴, R² and R¹ are the same as the preferences set out for the corresponding substituents of a compound of formula (I).

Another group of novel intermediates are compounds of formula (Int-II)

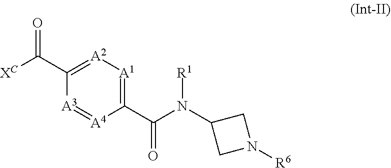

wherein A¹, A², A³, A⁴ and R¹ are as defined for a compound according to any one of embodiments 1 to 9; R⁶ is hydrogen or S(O)₂R² and X^{C} is CH₂-halogen, wherein halogen is e.g. bromo or chloro, CH═C(R³)R⁴ or CH₂C(OH)(R³)R⁴ wherein R³ and R⁴ are as defined for a compound of formula (I); and a salt or N-oxide thereof. The preferences for A¹, A², A³, A⁴, R², R³, R⁴ and R¹ are the same as the preferences set out for the corresponding substituents of a compound of formula (I).

Another group of novel intermediates are compounds of formula (Int-III)

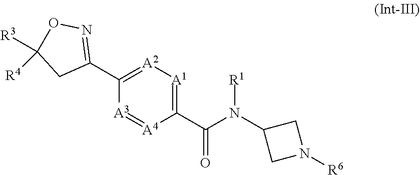

wherein A¹, A², A³, A⁴, R¹, R³ and R⁴ are as defined for a compound according to any one of embodiments 1 to 9 and R⁶ is hydrogen or an amine protecting group, such as a boc group (removable protecting groups are described in “Greene's Protective Groups in Organic Synthesis”, 4th ed., Wuts, P. G. M., Greene, T. W. 2007, J. Wiley, Hoboken, N.J.) and a salt or N-oxide thereof. The preferences for A¹, A², A³, A⁴, R¹, R³ and R⁴ are the same as the preferences set out for the corresponding substituents of a compound of formula (I).

The compounds of formula (I) and, where appropriate, the tautomer's thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula (I), in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents. Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

Compounds of formula (I) include at least one chiral center and may exist as compounds of formula (I*) or compounds of formula (I**):

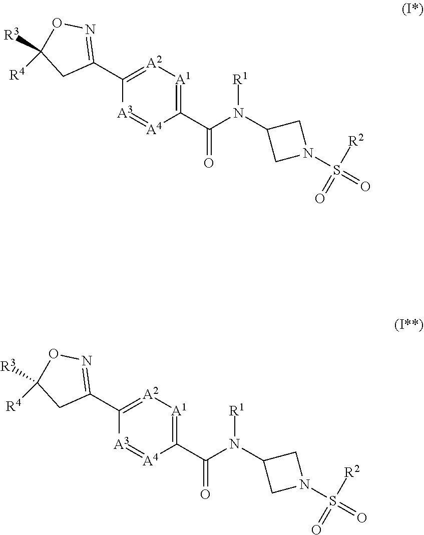

Generally compounds of formula (I**) are more biologically active than compounds of formula (I*). The invention includes mixtures of compounds (I*) and (I**) in any ratio e.g. in a molar ratio of 1:99 to 99:1, e.g. 10:1 to 1:10, e.g. a substantially 50:50 molar ratio. In an enantiomerically (or epimerically) enriched mixture of formula (I**), the molar proportion of compound (I**) compared to the total amount of both enantiomers (or epimers) is for example greater than 50%, e.g. at least 55, 60, 65, 70, 75, 80, 85, 90, 95, 96, 97, 98, or at least 99%. Likewise, in enantiomerically (or epimerically) enriched mixture of formula (I*), the molar proportion of the compound of formula (I*) compared to the total amount of both enantiomers (or epimers) is for example greater than 50%, e.g. at least 55, 60, 65, 70, 75, 80, 85, 90, 95, 96, 97, 98, or at least 99%. Enantiomerically (or epimerically) enriched mixtures of formula (I**) are preferred.

Salts of compounds of formula (I) can be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds (I), acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula (I), which have salt-forming properties, can be obtained in free form or in the form of salts.

N-oxides can be prepared by reacting a compound of the formula (I) with a suitable oxidizing agent, for example the H₂O₂/urea adduct in the presence of an acid anhydride, e.g. trifluoroacetic anhydride. Such oxidations are known from the literature, for example from J. Med. Chem. 1989, 32, 2561 or WO200015615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds according to the following Tables A-1 to A-210 below can be prepared according to the methods described above. The examples which follow are intended to illustrate the invention and show preferred compounds of formula (I).

Tables A-1 to A-210: Compounds of Formula (Ia)

The invention is further illustrated by making available the following individual compounds of formula (Ia) listed below in Tables A-1 to A-210.

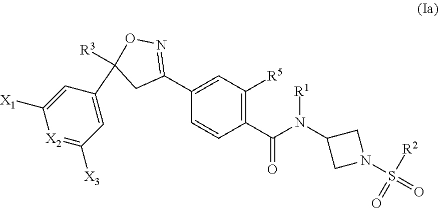

Each of Tables A-1 to A-210, which follow the Table B below, make available compounds of the formula (Ia) in which R², R³, R⁵ and X₁ are the substituents defined in Table B and R₁, X₃ and X₂ are the substituents defined in the relevant Table A-1 to A-210. Thus Table A-1 individualises compounds of formula (Ia) wherein for each row of Table B, the R², R³, R⁵ and X₁ substituents areas defined in Table A-1; similarly, Table A-2 individualises compounds of formula (Ia) wherein for each row of Table B, the R², R³, R⁵ and X₁ substituents are as defined in Table A-2; and so on for Tables A-3 to A-210. Each compound disclosed in Tables A-1 to A-210 represents a disclosure of a compound according to the compound of formula (I*), and a disclosure according to the compound of formula (I**) as well as mixtures thereof.

TABLE B
Substituent definitions of R², R³, R⁵ and X₁:

| Index | R³ | R⁵ | X₁ | R² |
|---|---|---|---|---|
| 1 | CF2Cl | Cl | Cl | CH3 |
| 2 | CF2Cl | Cl | Br | CH3 |
| 3 | CF2Cl | Cl | F | CH3 |
| 4 | CF2Cl | Cl | CF3 | CH3 |
| 5 | CF2Cl | Cl | H | CH3 |
| 6 | CF2Cl | Br | Cl | CH3 |
| 7 | CF2Cl | Br | Br | CH3 |
| 8 | CF2Cl | Br | F | CH3 |
| 9 | CF2Cl | Br | CF3 | CH3 |
| 10 | CF2Cl | Br | H | CH3 |
| 11 | CF2Cl | CF3 | Cl | CH3 |
| 12 | CF2Cl | CF3 | Br | CH3 |
| 13 | CF2Cl | CF3 | F | CH3 |
| 14 | CF2Cl | CF3 | CF3 | CH3 |
| 15 | CF2Cl | CF3 | H | CH3 |
| 16 | CF2Cl | CH3 | Cl | CH3 |
| 17 | CF2Cl | CH3 | Br | CH3 |
| 18 | CF2Cl | CH3 | F | CH3 |
| 19 | CF2Cl | CH3 | CF3 | CH3 |
| 20 | CF2Cl | CH3 | H | CH3 |
| 21 | CF2Cl | CH2CH3 | Cl | CH3 |
| 22 | CF2Cl | CH2CH3 | Br | CH3 |
| 23 | CF2Cl | CH2CH3 | F | CH3 |
| 24 | CF2Cl | CH2CH3 | CF3 | CH3 |
| 25 | CF2Cl | CH2CH3 | H | CH3 |
| 26 | CF3 | Cl | Cl | CH3 |
| 27 | CF3 | Cl | Br | CH3 |
| 28 | CF3 | Cl | F | CH3 |
| 29 | CF3 | Cl | CF3 | CH3 |
| 30 | CF3 | Cl | H | CH3 |
| 31 | CF3 | Br | Cl | CH3 |
| 32 | CF3 | Br | Br | CH3 |
| 33 | CF3 | Br | F | CH3 |
| 34 | CF3 | Br | CF3 | CH3 |
| 35 | CF3 | Br | H | CH3 |
| 36 | CF3 | CF3 | Cl | CH3 |
| 37 | CF3 | CF3 | Br | CH3 |
| 38 | CF3 | CF3 | F | CH3 |
| 39 | CF3 | CF3 | CF3 | CH3 |
| 40 | CF3 | CF3 | H | CH3 |
| 41 | CF3 | CH3 | Cl | CH3 |
| 42 | CF3 | CH3 | Br | CH3 |
| 43 | CF3 | CH3 | F | CH3 |
| 44 | CF3 | CH3 | CF3 | CH3 |
| 45 | CF3 | CH3 | H | CH3 |
| 46 | CF3 | CH2CH3 | Cl | CH3 |
| 47 | CF3 | CH2CH3 | Br | CH3 |
| 48 | CF3 | CH2CH3 | F | CH3 |
| 49 | CF3 | CH2CH3 | CF3 | CH3 |
| 50 | CF3 | CH2CH3 | H | CH3 |
| 51 | CF2Cl | Cl | Cl | CH2CH3 |
| 52 | CF2Cl | Cl | Br | CH2CH3 |
| 53 | CF2Cl | Cl | F | CH2CH3 |
| 54 | CF2Cl | Cl | CF3 | CH2CH3 |
| 55 | CF2Cl | Cl | H | CH2CH3 |
| 56 | CF2Cl | Br | Cl | CH2CH3 |
| 57 | CF2Cl | Br | Br | CH2CH3 |
| 58 | CF2Cl | Br | F | CH2CH3 |
| 59 | CF2Cl | Br | CF3 | CH2CH3 |
| 60 | CF2Cl | Br | H | CH2CH3 |
| 61 | CF2Cl | CF3 | Cl | CH2CH3 |
| 62 | CF2Cl | CF3 | Br | CH2CH3 |
| 63 | CF2Cl | CF3 | F | CH2CH3 |
| 64 | CF2Cl | CF3 | CF3 | CH2CH3 |
| 65 | CF2Cl | CF3 | H | CH2CH3 |
| 66 | CF2Cl | CH3 | Cl | CH2CH3 |
| 67 | CF2Cl | CH3 | Br | CH2CH3 |
| 68 | CF2Cl | CH3 | F | CH2CH3 |
| 69 | CF2Cl | CH3 | CF3 | CH2CH3 |
| 70 | CF2Cl | CH3 | H | CH2CH3 |
| 71 | CF2Cl | CH2CH3 | Cl | CH2CH3 |
| 72 | CF2Cl | CH2CH3 | Br | CH2CH3 |
| 73 | CF2Cl | CH2CH3 | F | CH2CH3 |
| 74 | CF2Cl | CH2CH3 | CF3 | CH2CH3 |
| 75 | CF2Cl | CH2CH3 | H | CH2CH3 |
| 76 | CF3 | Cl | Cl | CH2CH3 |
| 77 | CF3 | Cl | Br | CH2CH3 |
| 78 | CF3 | Cl | F | CH2CH3 |
| 79 | CF3 | Cl | CF3 | CH2CH3 |
| 80 | CF3 | Cl | H | CH2CH3 |
| 81 | CF3 | Br | Cl | CH2CH3 |
| 82 | CF3 | Br | Br | CH2CH3 |
| 83 | CF3 | Br | F | CH2CH3 |
| 84 | CF3 | Br | CF3 | CH2CH3 |
| 85 | CF3 | Br | H | CH2CH3 |
| 86 | CF3 | CF3 | Cl | CH2CH3 |
| 87 | CF3 | CF3 | Br | CH2CH3 |
| 88 | CF3 | CF3 | F | CH2CH3 |
| 89 | CF3 | CF3 | CF3 | CH2CH3 |
| 90 | CF3 | CF3 | H | CH2CH3 |
| 91 | CF3 | CH3 | Cl | CH2CH3 |
| 92 | CF3 | CH3 | Br | CH2CH3 |
| 93 | CF3 | CH3 | F | CH2CH3 |
| 94 | CF3 | CH3 | CF3 | CH2CH3 |
| 95 | CF3 | CH3 | H | CH2CH3 |
| 96 | CF3 | CH2CH3 | Cl | CH2CH3 |
| 97 | CF3 | CH2CH3 | Br | CH2CH3 |
| 98 | CF3 | CH2CH3 | F | CH2CH3 |
| 99 | CF3 | CH2CH3 | CF3 | CH2CH3 |
| 100 | CF3 | CH2CH3 | H | CH2CH3 |
| 101 | CF2Cl | Cl | Cl | CH2F |
| 102 | CF2Cl | Cl | Br | CH2F |
| 103 | CF2Cl | Cl | F | CH2F |
| 104 | CF2Cl | Cl | CF3 | CH2F |
| 105 | CF2Cl | Cl | H | CH2F |
| 106 | CF2Cl | Br | Cl | CH2F |
| 107 | CF2Cl | Br | Br | CH2F |
| 108 | CF2Cl | Br | F | CH2F |
| 109 | CF2Cl | Br | CF3 | CH2F |
| 110 | CF2Cl | Br | H | CH2F |
| 111 | CF2Cl | CF3 | Cl | CH2F |
| 112 | CF2Cl | CF3 | Br | CH2F |
| 113 | CF2Cl | CF3 | F | CH2F |
| 114 | CF2Cl | CF3 | CF3 | CH2F |
| 115 | CF2Cl | CF3 | H | CH2F |
| 116 | CF2Cl | CH3 | Cl | CH2F |
| 117 | CF2Cl | CH3 | Br | CH2F |
| 118 | CF2Cl | CH3 | F | CH2F |
| 119 | CF2Cl | CH3 | CF3 | CH2F |
| 120 | CF2Cl | CH3 | H | CH2F |
| 121 | CF2Cl | CH2CH3 | Cl | CH2F |
| 122 | CF2Cl | CH2CH3 | Br | CH2F |
| 123 | CF2Cl | CH2CH3 | F | CH2F |
| 124 | CF2Cl | CH2CH3 | CF3 | CH2F |
| 125 | CF2Cl | CH2CH3 | H | CH2F |
| 126 | CF3 | Cl | Cl | CH2F |
| 127 | CF3 | Cl | Br | CH2F |
| 128 | CF3 | Cl | F | CH2F |
| 129 | CF3 | Cl | CF3 | CH2F |
| 130 | CF3 | Cl | H | CH2F |
| 131 | CF3 | Br | Cl | CH2F |
| 132 | CF3 | Br | Br | CH2F |
| 133 | CF3 | Br | F | CH2F |
| 134 | CF3 | Br | CF3 | CH2F |
| 135 | CF3 | Br | H | CH2F |
| 136 | CF3 | CF3 | Cl | CH2F |
| 137 | CF3 | CF3 | Br | CH2F |
| 138 | CF3 | CF3 | F | CH2F |
| 139 | CF3 | CF3 | CF3 | CH2F |
| 140 | CF3 | CF3 | H | CH2F |
| 141 | CF3 | CH3 | Cl | CH2F |
| 142 | CF3 | CH3 | Br | CH2F |
| 143 | CF3 | CH3 | F | CH2F |
| 144 | CF3 | CH3 | CF3 | CH2F |
| 145 | CF3 | CH3 | H | CH2F |
| 146 | CF3 | CH2CH3 | Cl | CH2F |
| 147 | CF3 | CH2CH3 | Br | CH2F |
| 148 | CF3 | CH2CH3 | F | CH2F |
| 149 | CF3 | CH2CH3 | CF3 | CH2F |
| 150 | CF3 | CH2CH3 | H | CH2F |
| 151 | CF2Cl | Cl | Cl | CHF2 |
| 152 | CF2Cl | Cl | Br | CHF2 |
| 153 | CF2Cl | Cl | F | CHF2 |
| 154 | CF2Cl | Cl | CF3 | CHF2 |
| 155 | CF2Cl | Cl | H | CHF2 |
| 156 | CF2Cl | Br | Cl | CHF2 |
| 157 | CF2Cl | Br | Br | CHF2 |
| 158 | CF2Cl | Br | F | CHF2 |
| 159 | CF2Cl | Br | CF3 | CHF2 |
| 160 | CF2Cl | Br | H | CHF2 |
| 161 | CF2Cl | CF3 | Cl | CHF2 |
| 162 | CF2Cl | CF3 | Br | CHF2 |
| 163 | CF2Cl | CF3 | F | CHF2 |
| 164 | CF2Cl | CF3 | CF3 | CHF2 |
| 165 | CF2Cl | CF3 | H | CHF2 |
| 166 | CF2Cl | CH3 | Cl | CHF2 |
| 167 | CF2Cl | CH3 | Br | CHF2 |
| 168 | CF2Cl | CH3 | F | CHF2 |
| 169 | CF2Cl | CH3 | CF3 | CHF2 |
| 170 | CF2Cl | CH3 | H | CHF2 |
| 171 | CF2Cl | CH2CH3 | Cl | CHF2 |
| 172 | CF2Cl | CH2CH3 | Br | CHF2 |
| 173 | CF2Cl | CH2CH3 | F | CHF2 |
| 174 | CF2Cl | CH2CH3 | CF3 | CHF2 |
| 175 | CF2Cl | CH2CH3 | H | CHF2 |
| 176 | CF3 | Cl | Cl | CHF2 |
| 177 | CF3 | Cl | Br | CHF2 |
| 178 | CF3 | Cl | F | CHF2 |
| 179 | CF3 | Cl | CF3 | CHF2 |
| 180 | CF3 | Cl | H | CHF2 |
| 181 | CF3 | Br | Cl | CHF2 |
| 182 | CF3 | Br | Br | CHF2 |
| 183 | CF3 | Br | F | CHF2 |
| 184 | CF3 | Br | CF3 | CHF2 |
| 185 | CF3 | Br | H | CHF2 |
| 186 | CF3 | CF3 | Cl | CHF2 |
| 187 | CF3 | CF3 | Br | CHF2 |
| 188 | CF3 | CF3 | F | CHF2 |
| 189 | CF3 | CF3 | CF3 | CHF2 |
| 190 | CF3 | CF3 | H | CHF2 |
| 191 | CF3 | CH2CH3 | Cl | CHF2 |
| 192 | CF3 | CH2CH3 | Br | CHF2 |
| 193 | CF3 | CH2CH3 | F | CHF2 |
| 194 | CF3 | CH2CH3 | CF3 | CHF2 |
| 199 | CF3 | CH2CH3 | H | CHF2 |

Table A-1 provides 200 compounds A-1.001 to A-1.200 of formula (Ia) wherein R¹ is CH2O(C═O)OCH3, X2 is CF, X3 is Cl and R2, R3, R5, X1 are as defined in table B.

Table A-2 provides 200 compounds A-2.001 to A-2.200 of formula (Ia) wherein R1 is CH2(C═O)OCH3, X2 is CF, X3 is Br and R2, R3, R5, X1 are as defined in table B.

Table A-3 provides 200 compounds A-3.001 to A-3.200 of formula (Ia) wherein R1 is CH2O(C═O)OCH3, X2 is CF, X3 is F and R2, R3, R5, X1 are as defined in table B.

Table A-4 provides 200 compounds A-4.001 to A-4.200 of formula (Ia) wherein R1 is CH2O(C═O)OCH3, X2 is CF, X3 is CF₃ and R2, R3, R5, X1 are as defined in table B.

Table A-5 provides 200 compounds A-5.001 to A-5.200 of formula (Ia) wherein R1 is CH2O(C═O)OCH3, X2 is CF, X3 is H and R2, R3, R5, X1 are as defined in table B.

Table A-6 provides 200 compounds A-6.001 to A-6.200 of formula (Ia) wherein R1 is CH2O(C═O)OCH3, X2 is CCl, X3 is CI and R2, R3, R5, X1 are as defined in table B.

Table A-7 provides 200 compounds A-7.001 to A-7.200 of formula (Ia) wherein R1 is CH2O(C═O)OCH3, X2 is CCl, X3 is Br and R2, R3, R5, X1 are as defined in table B.

Table A-8 provides 200 compounds A-8.001 to A-8.200 of formula (Ia) wherein R1 is CH2O (C═O)OCH3, X2 is CCl, X3 is F and R2, R3, R5, X1 are as defined in table B.

Table A-9 provides 200 compounds A-9.001 to A-9.200 of formula (Ia) wherein R1 is CH2O(C═O)OCH3, X2 is CCl, X3 is CF₃ and R2, R3, R5, X1 are as defined in table B.

Table A-10 provides 200 compounds A-10.001 to A-10.200 of formula (Ia) wherein R1 is CH2O(C═O)OCH3, X2 is CCl, X3 is H and R2, R3, R5, X1 are as defined in table B.

Table A-11 provides 200 compounds A-11.001 to A-11.200 of formula (Ia) wherein R1 is CH2O(C═O)OCH3, X2 is CBr, X3 is CI and R2, R3, R5, X1 are as defined in table B.

Table A-12 provides 200 compounds A-12.001 to A-12.200 of formula (Ia) wherein R1 is CH2O(C═O)OCH3, X2 is CBr, X3 is Br and R2, R3, R5, X1 are as defined in table B.

Table A-13 provides 200 compounds A-13.001 to A-13.200 of formula (Ia) wherein R1 is CH2O(C═O)OCH3, X2 is CBr, X3 is F and R2, R3, R5, X1 are as defined in table B.

Table A-14 provides 200 compounds A-14.001 to A-14.200 of formula (Ia) wherein R1 is CH2O(C═O)OCH3, X2 is CBr, X3 is CF₃ and R2, R3, R5, X1 are as defined in table B.

Table A-15 provides 200 compounds A-15.001 to A-15.200 of formula (Ia) wherein R1 is CH2O(C═O)OCH3, X2 is CBr, X3 is H and R2, R3, R5, X1 are as defined in table B.

Table A-16 provides 200 compounds A-16.001 to A-16.200 of formula (Ia) wherein R1 is CH2O(C═O)OCH3, X2 is N, X3 is CI and R2, R3, R5, X1 are as defined in table B.

Table A-17 provides 200 compounds A-17.001 to A-17.200 of formula (Ia) wherein R1 is CH2O(C═O)OCH3, X2 is N, X3 is Br and R2, R3, R5, X1 are as defined in table B.

Table A-18 provides 200 compounds A-18.001 to A-18.200 of formula (Ia) wherein R1 is CH2O(C═O)OCH3, X2 is N, X3 is F and R2, R3, R5, X1 are as defined in table B.

Table A-19 provides 200 compounds A-19.001 to A-19.200 of formula (Ia) wherein R1 is CH2O(C═O)OCH3, X2 is N, X3 is CF₃ and R2, R3, R5, X1 are as defined in table B.

Table A-20 provides 200 compounds A-20.001 to A-20.200 of formula (Ia) wherein R1 is CH2O(C═O)OCH3, X2 is N, X3 is H and R2, R3, R5, X1 are as defined in table B.

Table A-21 provides 200 compounds A-21.001 to A-21.200 of formula (Ia) wherein R1 is CH2O(C═O)OCH3, X2 is CCF3, X3 is Cl and R2, R3, R5, X1 are as defined in table B.

Table A-22 provides 200 compounds A-22.001 to A-22.200 of formula (Ia) wherein R1 is CH2O(C═O)OCH3, X2 is CCF3, X3 is Br and R2, R3, R5, X1 are as defined in table B.

Table A-23 provides 200 compounds A-23.001 to A-23.200 of formula (Ia) wherein R1 is CH2O(C═O)OCH3, X2 is CCF3, X3 is F and R2, R3, R5, X1 are as defined in table B.

Table A-24 provides 200 compounds A-24.001 to A-24.200 of formula (Ia) wherein R1 is CH2O(C═O)OCH3, X2 is CCF3, X3 is CF₃ and R2, R3, R5, X1 are as defined in table B.

Table A-25 provides 200 compounds A-25.001 to A-25.200 of formula (Ia) wherein R1 is CH2O(C═O)OCH3, X2 is CCF3, X3 is H and R2, R3, R5, X1 are as defined in table B.

Table A-26 provides 200 compounds A-26.001 to A-26.200 of formula (Ia) wherein R1 is CH2O(C═O)OCH3, X2 is CH, X3 is Cl and R2, R3, R5, X1 are as defined in table B.

Table A-27 provides 200 compounds A-27.001 to A-27.200 of formula (Ia) wherein R1 is CH2O(C═O)OCH3, X2 is CH, X3 is Br and R2, R3, R5, X1 are as defined in table B.

Table A-28 provides 200 compounds A-28.001 to A-28.200 of formula (Ia) wherein R1 is CH2O(C═O)OCH3, X2 is CH, X3 is F and R2, R3, R5, X1 are as defined in table B.

Table A-29 provides 200 compounds A-29.001 to A-29.200 of formula (Ia) wherein R1 is CH2O(C═O)OCH3, X2 is CH, X3 is CF₃ and R2, R3, R5, X1 are as defined in table B.

Table A-30 provides 200 compounds A-30.001 to A-30.200 of formula (Ia) wherein R1 is CH2O (C═O)OCH3, X2 is CH, X3 is H and R2, R3, R5, X1 are as defined in table B.

Table A-31 provides 200 compounds A-31.001 to A-31.200 of formula (Ia) wherein R1 is CH2N(CH3) (C═O)OCH3, X2 is CF, X3 is Cl and R2, R3, R5, X1 are as defined in table B.

Table A-32 provides 200 compounds A-32.001 to A-32.200 of formula (Ia) wherein R1 is CH2N(CH3) (C═O)OCH3, X2 is CF, X3 is Br and R2, R3, R5, X1 are as defined in table B.

Table A-33 provides 200 compounds A-33.001 to A-33.200 of formula (Ia) wherein R1 is CH2N(CH3) (C═O)OCH3, X2 is CF, X3 is F and R2, R3, R5, X1 are as defined in table B.

Table A-34 provides 200 compounds A-34.001 to A-34.200 of formula (Ia) wherein R1 is CH2N(CH3) (C═O)OCH3, X2 is CF, X3 is CF₃ and R2, R3, R5, X1 are as defined in table B.

Table A-35 provides 200 compounds A-35.001 to A-35.200 of formula (Ia) wherein R1 is CH2N(CH3) (C═O)OCH3, X2 is CF, X3 is H and R2, R3, R5, X1 are as defined in table B.

Table A-36 provides 200 compounds A-36.001 to A-36.200 of formula (Ia) wherein R1 is CH2N(CH3) (C═O)OCH3, X2 is CCl, X3 is Cl and R2, R3, R5, X1 are as defined in table B.

Table A-37 provides 200 compounds A-37.001 to A-37.200 of formula (Ia) wherein R1 is CH2N(CH3) (C═O)OCH3, X2 is CC, X3 is Br and R2, R3, R5, X1 are as defined in table B.

Table A-38 provides 200 compounds A-38.001 to A-38.200 of formula (Ia) wherein R1 is CH2N(CH3) (C═O)OCH3, X2 is CC, X3 is F and R2, R3, R5, X1 are as defined in table B.

Table A-39 provides 200 compounds A-39.001 to A-39.200 of formula (Ia) wherein R1 is CH2N(CH3) (C═O)OCH3, X2 is CC, X3 is CF₃ and R2, R3, R5, X1 are as defined in table B.

Table A-40 provides 200 compounds A-40.001 to A-40.200 of formula (Ia) wherein R1 is CH2N(CH3) (C═O)OCH3, X2 is CCl, X3 is H and R2, R3, R5, X1 are as defined in table B.

Table A-41 provides 200 compounds A-41.001 to A-41.200 of formula (Ia) wherein R1 is CH2N(CH3) (C═O)OCH3, X2 is CBr, X3 is Cl and R2, R3, R5, X1 are as defined in table B.

Table A-42 provides 200 compounds A-42.001 to A-42.200 of formula (Ia) wherein R1 is CH2N(CH3) (C═O)OCH3, X2 is CBr, X3 is Br and R2, R3, R5, X1 are as defined in table B.

Table A-43 provides 200 compounds A-43.001 to A-43.200 of formula (Ia) wherein R1 is CH2N(CH3) (C═O)OCH3, X2 is CBr, X3 is F and R2, R3, R5, X1 are as defined in table B.

Table A-44 provides 200 compounds A-44.001 to A-44.200 of formula (Ia) wherein R1 is CH2N(CH3) (C═O)OCH3, X2 is CBr, X3 is CF₃ and R2, R3, R5, X1 are as defined in table B.

Table A-45 provides 200 compounds A-45.001 to A-45.200 of formula (Ia) wherein R1 is CH2N(CH3) (C═O)OCH3, X2 is CBr, X3 is H and R2, R3, R5, X1 are as defined in table B.

Table A-46 provides 200 compounds A-46.001 to A-46.200 of formula (Ia) wherein R1 is CH2N(CH3) (C═O)OCH3, X2 is N, X3 is C and R2, R3, R5, X1 are as defined in table B.

Table A-47 provides 200 compounds A-47.001 to A-47.200 of formula (Ia) wherein R1 is CH2N(CH3) (C═O)OCH3, X2 is N, X3 is Br and R2, R3, R5, X1 are as defined in table B.

Table A-48 provides 200 compounds A-48.001 to A-48.200 of formula (Ia) wherein R1 is CH2N(CH3) (C═O)OCH3, X2 is N, X3 is F and R2, R3, R5, X1 are as defined in table B.

Table A-49 provides 200 compounds A-49.001 to A-49.200 of formula (Ia) wherein R1 is CH2N(CH3) (C═O)OCH3, X2 is N, X3 is CF₃ and R2, R3, R5, X1 are as defined in table B.

Table A-50 provides 200 compounds A-50.001 to A-50.200 of formula (Ia) wherein R1 is CH2N(CH3) (C═O)OCH3, X2 is N, X3 is H and R2, R3, R5, X1 are as defined in table B.

Table A-51 provides 200 compounds A-51.001 to A-51.200 of formula (Ia) wherein R1 is CH2N(CH3) (C═O)OCH3, X2 is CCF3, X3 is C and R2, R3, R5, X1 are as defined in table B.

Table A-52 provides 200 compounds A-52.001 to A-52.200 of formula (Ia) wherein R1 is CH2N(CH3) (C═O)OCH3, X2 is CCF3, X3 is Br and R2, R3, R5, X1 are as defined in table B.

Table A-53 provides 200 compounds A-53.001 to A-53.200 of formula (Ia) wherein R1 is CH2N(CH3) (C═O)OCH3, X2 is CCF3, X3 is F and R2, R3, R5, X1 are as defined in table B.

Table A-54 provides 200 compounds A-54.001 to A-54.200 of formula (Ia) wherein R1 is CH2N(CH3) (C═O)OCH3, X2 is CCF3, X3 is CF₃ and R2, R3, R5, X1 are as defined in table B.

Table A-55 provides 200 compounds A-55.001 to A-55.200 of formula (Ia) wherein R1 is CH2N(CH3) (C═O)OCH3, X2 is CCF3, X3 is H and R2, R3, R5, X1 are as defined in table B.

Table A-56 provides 200 compounds A-56.001 to A-56.200 of formula (Ia) wherein R1 is CH2N(CH3) (C═O)OCH3, X2 is CH, X3 is Cl and R2, R3, R5, X1 are as defined in table B.

Table A-57 provides 200 compounds A-57.001 to A-57.200 of formula (Ia) wherein R1 is CH2N(CH3) (C═O)OCH3, X2 is CH, X3 is Br and R2, R3, R5, X1 are as defined in table B.

Table A-58 provides 200 compounds A-58.001 to A-58.200 of formula (Ia) wherein R1 is CH2N(CH3) (C═O)OCH3, X2 is CH, X3 is F and R2, R3, R5, X1 are as defined in table B.

Table A-59 provides 200 compounds A-59.001 to A-59.200 of formula (Ia) wherein R1 is CH2N(CH3) (C═O)OCH3, X2 is CH, X3 is CF₃ and R2, R3, R5, X1 are as defined in table B.

Table A-60 provides 200 compounds A-60.001 to A-60.200 of formula (Ia) wherein R1 is CH2N(CH3) (C═O)OCH3, X2 is CH, X3 is H and R2, R3, R5, X1 are as defined in table B.

Table A-61 provides 200 compounds A-61.001 to A-61.200 of formula (Ia) wherein R1 is CH2O(C═O)OCH2CH3, X2 is CF, X3 is Cl and R2, R3, R5, X1 are as defined in table B.

Table A-62 provides 200 compounds A-62.001 to A-62.200 of formula (Ia) wherein R1 is CH2O(C═O)OCH2CH3, X2 is CF, X3 is Br and R2, R3, R5, X1 are as defined in table B.

Table A-63 provides 200 compounds A-63.001 to A-63.200 of formula (Ia) wherein R1 is CH2O(C═O)OCH2CH3, X2 is CF, X3 is F and R2, R3, R5, X1 are as defined in table B.

Table A-64 provides 200 compounds A-64.001 to A-64.200 of formula (Ia) wherein R1 is CH2O (C═O)OCH2CH3, X2 is CF, X3 is CF₃ and R2, R3, R5, X1 are as defined in table B.

Table A-65 provides 200 compounds A-65.001 to A-65.200 of formula (Ia) wherein R1 is CH2O (C═O)OCH2CH3, X2 is CF, X3 is H and R2, R3, R5, X1 are as defined in table B.

Table A-66 provides 200 compounds A-66.001 to A-66.200 of formula (Ia) wherein R1 is CH2O (C═O)OCH2CH3, X2 is CCl, X3 is Cl and R2, R3, R5, X1 are as defined in table B.

Table A-67 provides 200 compounds A-67.001 to A-67.200 of formula (Ia) wherein R1 is CH2O (C═O)OCH2CH3, X2 is CCl, X3 is Br and R2, R3, R5, X1 are as defined in table B.

Table A-68 provides 200 compounds A-68.001 to A-68.200 of formula (Ia) wherein R1 is CH2O(C═O)OCH2CH3, X2 is CCl, X3 is F and R2, R3, R5, X1 are as defined in table B.

Table A-69 provides 200 compounds A-69.001 to A-69.200 of formula (Ia) wherein R1 is CH2O (C═O)OCH2CH3, X2 is CCl, X3 is CF₃ and R2, R3, R5, X1 are as defined in table B.

Table A-70 provides 200 compounds A-70.001 to A-70.200 of formula (Ia) wherein R1 is CH2O(C═O)OCH2CH3, X2 is CCl, X3 is H and R2, R3, R5, X1 are as defined in table B.

Table A-71 provides 200 compounds A-71.001 to A-71.200 of formula (Ia) wherein R1 is CH2O(C═O)OCH2CH3, X2 is CBr, X3 is CI and R2, R3, R5, X1 are as defined in table B.

Table A-72 provides 200 compounds A-72.001 to A-72.200 of formula (Ia) wherein R1 is CH2O(C═O)OCH2CH3, X2 is CBr, X3 is Br and R2, R3, R5, X1 are as defined in table B.

Table A-73 provides 200 compounds A-73.001 to A-73.200 of formula (Ia) wherein R1 is CH2O(C═O)OCH2CH3, X2 is CBr, X3 is F and R2, R3, R5, X1 are as defined in table B.

Table A-74 provides 200 compounds A-74.001 to A-74.200 of formula (Ia) wherein R1 is CH2O(C═O)OCH2CH3, X2 is CBr, X3 is CF₃ and R2, R3, R5, X1 are as defined in table B.

Table A-75 provides 200 compounds A-75.001 to A-75.200 of formula (Ia) wherein R1 is CH2O(C═O)OCH2CH3, X2 is CBr, X3 is H and R2, R3, R5, X1 are as defined in table B.

Table A-76 provides 200 compounds A-76.001 to A-76.200 of formula (Ia) wherein R1 is CH2O(C═O)OCH2CH3, X2 is N, X3 is Cl and R2, R3, R5, X1 are as defined in table B.

Table A-77 provides 200 compounds A-77.001 to A-77.200 of formula (Ia) wherein R1 is CH2O(C═O)OCH2CH3, X2 is N, X3 is Br and R2, R3, R5, X1 are as defined in table B.

Table A-78 provides 200 compounds A-78.001 to A-78.200 of formula (Ia) wherein R1 is CH2O(C═O)OCH2CH3, X2 is N, X3 is F and R2, R3, R5, X1 are as defined in table B.

Table A-79 provides 200 compounds A-79.001 to A-79.200 of formula (Ia) wherein R1 is CH2O(C═O)OCH2CH3, X2 is N, X3 is CF₃ and R2, R3, R5, X1 are as defined in table B.

Table A-80 provides 200 compounds A-80.001 to A-80.200 of formula (Ia) wherein R1 is CH2O(C═O)OCH2CH3, X2 is N, X3 is H and R2, R3, R5, X1 are as defined in table B.

Table A-81 provides 200 compounds A-81.001 to A-81.200 of formula (Ia) wherein R1 is CH2O(C═O)OCH2CH3, X2 is CCF3, X3 is Cl and R2, R3, R5, X1 are as defined in table B.

Table A-82 provides 200 compounds A-82.001 to A-82.200 of formula (Ia) wherein R1 is CH2O(C═O)OCH2CH3, X2 is CCF3, X3 is Br and R2, R3, R5, X1 are as defined in table B.

Table A-83 provides 200 compounds A-83.001 to A-83.200 of formula (Ia) wherein R1 is CH2O(C═O)OCH2CH3, X2 is CCF3, X3 is F and R2, R3, R5, X1 are as defined in table B.

Table A-84 provides 200 compounds A-84.001 to A-84.200 of formula (Ia) wherein R1 is CH2O(C═O)OCH2CH3, X2 is CCF3, X3 is CF₃ and R2, R3, R5, X1 are as defined in table B.

Table A-85 provides 200 compounds A-85.001 to A-85.200 of formula (Ia) wherein R1 is CH2O(C═O)OCH2CH3, X2 is CCF3, X3 is H and R2, R3, R5, X1 are as defined in table B.

Table A-86 provides 200 compounds A-86.001 to A-86.200 of formula (Ia) wherein R1 is CH2O(C═O)OCH2CH3, X2 is CH, X3 is Cl and R2, R3, R5, X1 are as defined in table B.

Table A-87 provides 200 compounds A-87.001 to A-87.200 of formula (Ia) wherein R1 is CH2O(C═O)OCH2CH3, X2 is CH, X3 is Br and R2, R3, R5, X1 are as defined in table B.

Table A-88 provides 200 compounds A-88.001 to A-88.200 of formula (Ia) wherein R1 is CH2O(C═O)OCH2CH3, X2 is CH, X3 is F and R2, R3, R5, X1 are as defined in table B.

Table A-89 provides 200 compounds A-89.001 to A-89.200 of formula (Ia) wherein R1 is CH2O(C═O)OCH2CH3, X2 is CH, X3 is CF₃ and R2, R3, R5, X1 are as defined in table B.

Table A-90 provides 200 compounds A-90.001 to A-90.200 of formula (Ia) wherein R1 is CH2O(C═O)OCH2CH3, X2 is CH, X3 is H and R2, R3, R5, X1 are as defined in table B.

Table A-91 provides 200 compounds A-91.001 to A-91.200 of formula (Ia) wherein R1 is CH2O(C═O)CH3, X2 is CF, X3 is Cl and R2, R3, R5, X1 are as defined in table B.

Table A-92 provides 200 compounds A-92.001 to A-92.200 of formula (Ia) wherein R1 is CH2O(C═O)CH3, X2 is CF, X3 is Br and R2, R3, R5, X1 are as defined in table B.

Table A-93 provides 200 compounds A-93.001 to A-93.200 of formula (Ia) wherein R1 is CH2O(C═O)CH3, X2 is CF, X3 is F and R2, R3, R5, X1 are as defined in table B.

Table A-94 provides 200 compounds A-94.001 to A-94.200 of formula (Ia) wherein R1 is CH2O(C═O)CH3, X2 is CF, X3 is CF₃ and R2, R3, R5, X1 are as defined in table B.

Table A-95 provides 200 compounds A-95.001 to A-95.200 of formula (Ia) wherein R1 is CH2O(C═O)CH3, X2 is CF, X3 is H and R2, R3, R5, X1 are as defined in table B.

Table A-96 provides 200 compounds A-96.001 to A-96.200 of formula (Ia) wherein R1 is CH2O (C═O)CH3, X2 is CC, X3 is C and R2, R3, R5, X1 are as defined in table B.

Table A-97 provides 200 compounds A-97.001 to A-97.200 of formula (Ia) wherein R1 is CH2O(C═O)CH3, X2 is CCl, X3 is Br and R2, R3, R5, X1 are as defined in table B.

Table A-98 provides 200 compounds A-98.001 to A-98.200 of formula (Ia) wherein R1 is CH2O (C═O)CH3, X2 is CC, X3 is F and R2, R3, R5, X1 are as defined in table B.

Table A-99 provides 200 compounds A-99.001 to A-99.200 of formula (a) wherein R1 is CH2O (C═O)CH3, X2 is CC, X3 is CF₃ and R2, R3, R5, X1 are as defined in table B.

Table A-100 provides 200 compounds A-100.001 to A-100.200 of formula (Ia) wherein R1 is CH2O(C═O)CH3, X2 is CCl, X3 is H and R2, R3, R5, X1 are as defined in table B.

Table A-101 provides 200 compounds A-101.001 to A-101.200 of formula (Ia) wherein R1 is CH2O(C═O)CH3, X2 is CBr, X3 is C and R2, R3, R5, X1 are as defined in table B.

Table A-102 provides 200 compounds A-102.001 to A-102.200 of formula (Ia) wherein R1 is CH2O (C═O)CH3, X2 is CBr, X3 is Br and R2, R3, R5, X1 are as defined in table B.

Table A-103 provides 200 compounds A-103.001 to A-103.200 of formula (Ia) wherein R1 is CH2O (C═O)CH3, X2 is CBr, X3 is F and R2, R3, R5, X1 are as defined in table B.

Table A-104 provides 200 compounds A-104.001 to A-104.200 of formula (Ia) wherein R1 is CH2O (C═O)CH3, X2 is CBr, X3 is CF₃ and R2, R3, R5, X1 are as defined in table B.

Table A-105 provides 200 compounds A-105.001 to A-105.200 of formula (Ia) wherein R1 is CH2O (C═O)CH3, X2 is CBr, X3 is H and R2, R3, R5, X1 are as defined in table B.

Table A-106 provides 200 compounds A-106.001 to A-106.200 of formula (Ia) wherein R1 is CH2O (C═O)CH3, X2 is N, X3 is C and R2, R3, R5, X1 are as defined in table B.

Table A-107 provides 200 compounds A-107.001 to A-107.200 of formula (Ia) wherein R1 is CH2O (C═O)CH3, X2 is N, X3 is Br and R2, R3, R5, X1 are as defined in table B.

Table A-108 provides 200 compounds A-108.001 to A-108.200 of formula (Ia) wherein R1 is CH2O (C═O)CH3, X2 is N, X3 is F and R2, R3, R5, X1 are as defined in table B.

Table A-109 provides 200 compounds A-109.001 to A-109.200 of formula (Ia) wherein R1 is CH2O(C═O)CH3, X2 is N, X3 is CF₃ and R2, R3, R5, X1 are as defined in table B.

Table A-110 provides 200 compounds A-110.001 to A-110.200 of formula (Ia) wherein R1 is CH2O(C═O)CH3, X2 is N, X3 is H and R2, R3, R5, X1 are as defined in table B.

Table A-111 provides 200 compounds A-111.001 to A-111.200 of formula (Ia) wherein R1 is CH2O(C═O)CH3, X2 is CCF3, X3 is C and R2, R3, R5, X1 are as defined in table B.

Table A-112 provides 200 compounds A-112.001 to A-112.200 of formula (Ia) wherein R1 is CH2O(C═O)CH3, X2 is CCF3, X3 is Br and R2, R3, R5, X1 are as defined in table B.

Table A-113 provides 200 compounds A-113.001 to A-113.200 of formula (Ia) wherein R1 is CH2O (C═O)CH3, X2 is CCF3, X3 is F and R2, R3, R5, X1 are as defined in table B.

Table A-114 provides 200 compounds A-114.001 to A-114.200 of formula (Ia) wherein R1 is CH2O(C═O)CH3, X2 is CCF3, X3 is CF₃ and R2, R3, R5, X1 are as defined in table B.

Table A-115 provides 200 compounds A-115.001 to A-115.200 of formula (Ia) wherein R1 is CH2O(C═O)CH3, X2 is CCF3, X3 is H and R2, R3, R5, X1 are as defined in table B.

Table A-116 provides 200 compounds A-116.001 to A-116.200 of formula (Ia) wherein R1 is CH2O(C═O)CH3, X2 is CH, X3 is Cl and R2, R3, R5, X1 are as defined in table B.

Table A-117 provides 200 compounds A-117.001 to A-117.200 of formula (Ia) wherein R1 is CH2O (C═O)CH3, X2 is CH, X3 is Br and R2, R3, R5, X1 are as defined in table B.

Table A-118 provides 200 compounds A-118.001 to A-118.200 of formula (Ia) wherein R1 is CH2O(C═O)CH3, X2 is CH, X3 is F and R2, R3, R5, X1 are as defined in table B.

Table A-119 provides 200 compounds A-119.001 to A-119.200 of formula (Ia) wherein R1 is CH2O(C═O)CH3, X2 is CH, X3 is CF₃ and R2, R3, R5, X1 are as defined in table B.

Table A-120 provides 200 compounds A-120.001 to A-120.200 of formula (Ia) wherein R1 is CH2O(C═O)CH3, X2 is CH, X3 is H and R2, R3, R5, X1 are as defined in table B.

Table A-121 provides 200 compounds A-121.001 to A-121.200 of formula (Ia) wherein R1 is CH2O(C═O)CH(CH3)2, X2 is CF, X3 is C and R2, R3, R5, X1 are as defined in table B.

Table A-122 provides 200 compounds A-122.001 to A-122.200 of formula (Ia) wherein R1 is CH2O(C═O)CH(CH3)2, X2 is CF, X3 is Br and R2, R3, R5, X1 are as defined in table B.

Table A-123 provides 200 compounds A-123.001 to A-123.200 of formula (Ia) wherein R1 is CH2O (C═O)CH(CH3)2, X2 is CF, X3 is F and R2, R3, R5, X1 are as defined in table B.

Table A-124 provides 200 compounds A-124.001 to A-124.200 of formula (Ia) wherein R1 is CH2O (C═O)CH(CH3)2, X2 is CF, X3 is CF₃ and R2, R3, R5, X1 are as defined in table B.

Table A-125 provides 200 compounds A-125.001 to A-125.200 of formula (Ia) wherein R1 is CH2O (C═O)CH(CH3)2, X2 is CF, X3 is H and R2, R3, R5, X1 are as defined in table B.

Table A-126 provides 200 compounds A-126.001 to A-126.200 of formula (Ia) wherein R1 is CH2O(C═O)CH(CH3)2, X2 is CCl, X3 is C and R2, R3, R5, X1 are as defined in table B.

Table A-127 provides 200 compounds A-127.001 to A-127.200 of formula (Ia) wherein R1 is CH2O (C═O)CH(CH3)2, X2 is CCl, X3 is Br and R2, R3, R5, X1 are as defined in table B.

Table A-128 provides 200 compounds A-128.001 to A-128.200 of formula (Ia) wherein R1 is CH2O (C═O)CH(CH3)2, X2 is CC, X3 is F and R2, R3, R5, X1 are as defined in table B.

Table A-129 provides 200 compounds A-129.001 to A-129.200 of formula (Ia) wherein R1 is CH2O (C═O)CH(CH3)2, X2 is CC, X3 is CF₃ and R2, R3, R5, X1 are as defined in table B.

Table A-130 provides 200 compounds A-130.001 to A-130.200 of formula (Ia) wherein R1 is CH2O(C═O)CH(CH3)2, X2 is CCl, X3 is H and R2, R3, R5, X1 are as defined in table B.

Table A-131 provides 200 compounds A-131.001 to A-131.200 of formula (Ia) wherein R1 is CH2O(C═O)CH(CH3)2, X2 is CBr, X3 is CI and R2, R3, R5, X1 are as defined in table B.

Table A-132 provides 200 compounds A-132.001 to A-132.200 of formula (Ia) wherein R1 is CH2O(C═O)CH(CH3)2, X2 is CBr, X3 is Br and R2, R3, R5, X1 are as defined in table B.

Table A-133 provides 200 compounds A-133.001 to A-133.200 of formula (Ia) wherein R1 is CH2O(C═O)CH(CH3)2, X2 is CBr, X3 is F and R2, R3, R5, X1 are as defined in table B.

Table A-134 provides 200 compounds A-134.001 to A-134.200 of formula (Ia) wherein R1 is CH2O(C═O)CH(CH3)2, X2 is CBr, X3 is CF₃ and R2, R3, R5, X1 are as defined in table B.

Table A-135 provides 200 compounds A-135.001 to A-135.200 of formula (Ia) wherein R1 is CH2O(C═O)CH(CH3)2, X2 is CBr, X3 is H and R2, R3, R5, X1 are as defined in table B.

Table A-136 provides 200 compounds A-136.001 to A-136.200 of formula (Ia) wherein R1 is CH2O(C═O)CH(CH3)2, X2 is N, X3 is Cl and R2, R3, R5, X1 are as defined in table B.

Table A-137 provides 200 compounds A-137.001 to A-137.200 of formula (Ia) wherein R1 is CH2O(C═O)CH(CH3)2, X2 is N, X3 is Br and R2, R3, R5, X1 are as defined in table B.

Table A-138 provides 200 compounds A-138.001 to A-138.200 of formula (Ia) wherein R1 is CH2O(C═O)CH(CH3)2, X2 is N, X3 is F and R2, R3, R5, X1 are as defined in table B.

Table A-139 provides 200 compounds A-139.001 to A-139.200 of formula (Ia) wherein R1 is CH2O(C═O)CH(CH3)2, X2 is N, X3 is CF₃ and R2, R3, R5, X1 are as defined in table B.

Table A-140 provides 200 compounds A-140.001 to A-140.200 of formula (Ia) wherein R1 is CH2O(C═O)CH(CH3)2, X2 is N, X3 is H and R2, R3, R5, X1 are as defined in table B.

Table A-141 provides 200 compounds A-141.001 to A-141.200 of formula (Ia) wherein R1 is CH2O(C═O)CH(CH3)2, X2 is CCF3, X3 is CI and R2, R3, R5, X1 are as defined in table B.

Table A-142 provides 200 compounds A-142.001 to A-142.200 of formula (Ia) wherein R1 is CH2O(C═O)CH(CH3)2, X2 is CCF3, X3 is Br and R2, R3, R5, X1 are as defined in table B.

Table A-143 provides 200 compounds A-143.001 to A-143.200 of formula (Ia) wherein R1 is CH2O(C═O)CH(CH3)2, X2 is CCF3, X3 is F and R2, R3, R5, X1 are as defined in table B.

Table A-144 provides 200 compounds A-144.001 to A-144.200 of formula (Ia) wherein R1 is CH2O(C═O)CH(CH3)2, X2 is CCF3, X3 is CF₃ and R2, R3, R5, X1 are as defined in table B.

Table A-145 provides 200 compounds A-145.001 to A-145.200 of formula (Ia) wherein R1 is CH2O(C═O)CH(CH3)2, X2 is CCF3, X3 is H and R2, R3, R5, X1 are as defined in table B.

Table A-146 provides 200 compounds A-146.001 to A-146.200 of formula (Ia) wherein R1 is CH2O(C═O)CH(CH3)2, X2 is CH, X3 is CI and R2, R3, R5, X1 are as defined in table B.

Table A-147 provides 200 compounds A-147.001 to A-147.200 of formula (Ia) wherein R1 is CH2O(C═O)CH(CH3)2, X2 is CH, X3 is Br and R2, R3, R5, X1 are as defined in table B.

Table A-148 provides 200 compounds A-148.001 to A-148.200 of formula (Ia) wherein R1 is CH2O(C═O)CH(CH3)2, X2 is CH, X3 is F and R2, R3, R5, X1 are as defined in table B.

Table A-149 provides 200 compounds A-149.001 to A-149.200 of formula (Ia) wherein R1 is CH2O(C═O)CH(CH3)2, X2 is CH, X3 is CF₃ and R2, R3, R5, X1 are as defined in table B.

Table A-150 provides 200 compounds A-150.001 to A-150.200 of formula (Ia) wherein R1 is CH2O(C═O)CH(CH3)2, X2 is CH, X3 is H and R2, R3, R5, X1 are as defined in table B.

Table A-151 provides 200 compounds A-151.001 to A-151.200 of formula (Ia) wherein R1 is CH2O (C═O)CHcPr, X2 is CF, X3 is Cl and R2, R3, R5, X1 are as defined in table B.

Table A-152 provides 200 compounds A-152.001 to A-152.200 of formula (Ia) wherein R1 is CH2O(C═O)CHcPr, X2 is CF, X3 is Br and R2, R3, R5, X1 are as defined in table B.

Table A-153 provides 200 compounds A-153.001 to A-153.200 of formula (Ia) wherein R1 is CH2O (C═O)CHcPr, X2 is CF, X3 is F and R2, R3, R5, X1 are as defined in table B.

Table A-154 provides 200 compounds A-154.001 to A-154.200 of formula (Ia) wherein R1 is CH2O (C═O)CHcPr, X2 is CF, X3 is CF₃ and R2, R3, R5, X1 are as defined in table B.

Table A-155 provides 200 compounds A-155.001 to A-155.200 of formula (Ia) wherein R1 is CH2O(C═O)CHcPr, X2 is CF, X3 is H and R2, R3, R5, X1 are as defined in table B.

Table A-156 provides 200 compounds A-156.001 to A-156.200 of formula (Ia) wherein R1 is CH2O(C═O)CHcPr, X2 is CC, X3 is C₀ and R2, R3, R5, X1 are as defined in table B.

Table A-157 provides 200 compounds A-157.001 to A-157.200 of formula (Ia) wherein R1 is CH2O (C═O)CHcPr, X2 is CC, X3 is Br and R2, R3, R5, X1 are as defined in table B.

Table A-158 provides 200 compounds A-158.001 to A-158.200 of formula (Ia) wherein R1 is CH2O (C═O)CHcPr, X2 is CC, X3 is F and R2, R3, R5, X1 are as defined in table B.

Table A-159 provides 200 compounds A-159.001 to A-159.200 of formula (Ia) wherein R1 is CH2O (C═O)CHcPr, X2 is CCl, X3 is CF₃ and R2, R3, R5, X1 are as defined in table B.

Table A-160 provides 200 compounds A-160.001 to A-160.200 of formula (Ia) wherein R1 is CH2O (C═O)CHcPr, X2 is CC, X3 is H and R2, R3, R5, X1 are as defined in table B.

Table A-161 provides 200 compounds A-161.001 to A-161.200 of formula (Ia) wherein R1 is CH2O (C═O)CHcPr, X2 is CBr, X3 is Cl and R2, R3, R5, X1 are as defined in table B.

Table A-162 provides 200 compounds A-162.001 to A-162.200 of formula (Ia) wherein R1 is CH2O (C═O)CHcPr, X2 is CBr, X3 is Br and R2, R3, R5, X1 are as defined in table B.

Table A-163 provides 200 compounds A-163.001 to A-163.200 of formula (Ia) wherein R1 is CH2O (C═O)CHcPr, X2 is CBr, X3 is F and R2, R3, R5, X1 are as defined in table B.

Table A-164 provides 200 compounds A-164.001 to A-164.200 of formula (Ia) wherein R1 is CH2O (C═O)CHcPr, X2 is CBr, X3 is CF₃ and R2, R3, R5, X1 are as defined in table B.

Table A-165 provides 200 compounds A-165.001 to A-165.200 of formula (Ia) wherein R1 is CH2O (C═O)CHcPr, X2 is CBr, X3 is H and R2, R3, R5, X1 are as defined in table B.

Table A-166 provides 200 compounds A-166.001 to A-166.200 of formula (Ia) wherein R1 is CH2O(C═O)CHcPr, X2 is N, X3 is Cl and R2, R3, R5, X1 are as defined in table B.

Table A-167 provides 200 compounds A-167.001 to A-167.200 of formula (Ia) wherein R1 is CH2O (C═O)CHcPr, X2 is N, X3 is Br and R2, R3, R5, X1 are as defined in table B.

Table A-168 provides 200 compounds A-168.001 to A-168.200 of formula (Ia) wherein R1 is CH2O (C═O)CHcPr, X2 is N, X3 is F and R2, R3, R5, X1 are as defined in table B.

Table A-169 provides 200 compounds A-169.001 to A-169.200 of formula (Ia) wherein R1 is CH2O (C═O)CHcPr, X2 is N, X3 is CF₃ and R2, R3, R5, X1 are as defined in table B.

Table A-170 provides 200 compounds A-170.001 to A-170.200 of formula (Ia) wherein R1 is CH2O(C═O)CHcPr, X2 is N, X3 is H and R2, R3, R5, X1 are as defined in table B.

Table A-171 provides 200 compounds A-171.001 to A-171.200 of formula (Ia) wherein R1 is CH2O (C═O)CHcPr, X2 is CCF3, X3 is Cl and R2, R3, R5, X1 are as defined in table B.

Table A-172 provides 200 compounds A-172.001 to A-172.200 of formula (Ia) wherein R1 is CH2O(C═O)CHcPr, X2 is CCF3, X3 is Br and R2, R3, R5, X1 are as defined in table B.

Table A-173 provides 200 compounds A-173.001 to A-173.200 of formula (Ia) wherein R1 is CH2O (C═O)CHcPr, X2 is CCF3, X3 is F and R2, R3, R5, X1 are as defined in table B.

Table A-174 provides 200 compounds A-174.001 to A-174.200 of formula (Ia) wherein R1 is CH2O(C═O)CHcPr, X2 is CCF3, X3 is CF₃ and R2, R3, R5, X1 are as defined in table B.

Table A-175 provides 200 compounds A-175.001 to A-175.200 of formula (Ia) wherein R1 is CH2O(C═O)CHcPr, X2 is CCF3, X3 is H and R2, R3, R5, X1 are as defined in table B.

Table A-176 provides 200 compounds A-176.001 to A-176.200 of formula (Ia) wherein R1 is CH2O (C═O)CHcPr, X2 is CH, X3 is Cl and R2, R3, R5, X1 are as defined in table B.

Table A-177 provides 200 compounds A-177.001 to A-177.200 of formula (Ia) wherein R1 is CH2O (C═O)CHcPr, X2 is CH, X3 is Br and R2, R3, R5, X1 are as defined in table B.

Table A-178 provides 200 compounds A-178.001 to A-178.200 of formula (Ia) wherein R1 is CH2O (C═O)CHcPr, X2 is CH, X3 is F and R2, R3, R5, X1 are as defined in table B.

Table A-179 provides 200 compounds A-179.001 to A-179.200 of formula (Ia) wherein R1 is CH2O (C═O)CHcPr, X2 is CH, X3 is CF₃ and R2, R3, R5, X1 are as defined in table B.

Table A-180 provides 200 compounds A-180.001 to A-180.200 of formula (Ia) wherein R1 is CH2O(C═O)CHcPr, X2 is CH, X3 is H and R2, R3, R5, X1 are as defined in table B.

Table A-181 provides 200 compounds A-181.001 to A-181.200 of formula (Ia) wherein R1 is CH2O(C═O)CH(CH3)3, X2 is CF, X3 is C and R2, R3, R5, X1 are as defined in table B.

Table A-182 provides 200 compounds A-182.001 to A-182.200 of formula (Ia) wherein R1 is CH2O(C═O)CH(CH3)3, X2 is CF, X3 is Br and R2, R3, R5, X1 are as defined in table B.

Table A-183 provides 200 compounds A-183.001 to A-183.200 of formula (Ia) wherein R1 is CH2O(C═O)CH(CH3)3, X2 is CF, X3 is F and R2, R3, R5, X1 are as defined in table B.

Table A-184 provides 200 compounds A-184.001 to A-184.200 of formula (Ia) wherein R1 is CH2O (C═O)CH(CH3)3, X2 is CF, X3 is CF₃ and R2, R3, R5, X1 are as defined in table B.

Table A-185 provides 200 compounds A-185.001 to A-185.200 of formula (Ia) wherein R1 is CH2O (C═O)CH(CH3)3, X2 is CF, X3 is H and R2, R3, R5, X1 are as defined in table B.

Table A-186 provides 200 compounds A-186.001 to A-186.200 of formula (Ia) wherein R1 is CH2O(C═O)CH(CH3)3, X2 is CCl, X3 is C and R2, R3, R5, X1 are as defined in table B.

Table A-187 provides 200 compounds A-187.001 to A-187.200 of formula (Ia) wherein R1 is CH2O(C═O)CH(CH3)3, X2 is CC, X3 is Br and R2, R3, R5, X1 are as defined in table B.

Table A-188 provides 200 compounds A-188.001 to A-188.200 of formula (Ia) wherein R1 is CH2O(C═O)CH(CH3)3, X2 is CC, X3 is F and R2, R3, R5, X1 are as defined in table B.

Table A-189 provides 200 compounds A-189.001 to A-189.200 of formula (Ia) wherein R1 is CH2O (C═O)CH(CH3)3, X2 is CCl, X3 is CF₃ and R2, R3, R5, X1 are as defined in table B.

Table A-190 provides 200 compounds A-190.001 to A-190.200 of formula (Ia) wherein R1 is CH2O(C═O)CH(CH3)3, X2 is CCl, X3 is H and R2, R3, R5, X1 are as defined in table B.

Table A-191 provides 200 compounds A-191.001 to A-191.200 of formula (Ia) wherein R1 is CH2O(C═O)CH(CH3)3, X2 is CBr, X3 is CI and R2, R3, R5, X1 are as defined in table B.

Table A-192 provides 200 compounds A-192.001 to A-192.200 of formula (Ia) wherein R1 is CH2O(C═O)CH(CH3)3, X2 is CBr, X3 is Br and R2, R3, R5, X1 are as defined in table B.

Table A-193 provides 200 compounds A-193.001 to A-193.200 of formula (Ia) wherein R1 is CH2O(C═O)CH(CH3)3, X2 is CBr, X3 is F and R2, R3, R5, X1 are as defined in table B.

Table A-194 provides 200 compounds A-194.001 to A-194.200 of formula (Ia) wherein R1 is CH2O(C═O)CH(CH3)3, X2 is CBr, X3 is CF₃ and R2, R3, R5, X1 are as defined in table B.

Table A-195 provides 200 compounds A-195.001 to A-195.200 of formula (Ia) wherein R1 is CH2O(C═O)CH(CH3)3, X2 is CBr, X3 is H and R2, R3, R5, X1 are as defined in table B.

Table A-196 provides 200 compounds A-196.001 to A-196.200 of formula (Ia) wherein R1 is CH2O(C═O)CH(CH3)3, X2 is N, X3 is Cl and R2, R3, R5, X1 are as defined in table B.

Table A-197 provides 200 compounds A-197.001 to A-197.200 of formula (Ia) wherein R1 is CH2O(C═O)CH(CH3)3, X2 is N, X3 is Br and R2, R3, R5, X1 are as defined in table B.

Table A-198 provides 200 compounds A-198.001 to A-198.200 of formula (Ia) wherein R1 is CH2(C═O)CH(CH3)3, X2 is N, X3 is F and R2, R3, R5, X1 are as defined in table B.

Table A-199 provides 200 compounds A-199.001 to A-199.200 of formula (Ia) wherein R1 is CH2O(C═O)CH(CH3)3, X2 is N, X3 is CF₃ and R2, R3, R5, X1 are as defined in table B.

Table A-200 provides 200 compounds A-200.001 to A-200.200 of formula (Ia) wherein R1 is CH2O(C═O)CH(CH3)3, X2 is N, X3 is H and R2, R3, R5, X1 are as defined in table B.

Table A-201 provides 200 compounds A-201.001 to A-201.200 of formula (Ia) wherein R1 is CH2O(C═O)CH(CH3)3, X2 is CCF3, X3 is CI and R2, R3, R5, X1 are as defined in table B.

Table A-202 provides 200 compounds A-202.001 to A-202.200 of formula (Ia) wherein R1 is CH2O(C═O)CH(CH3)3, X2 is CCF3, X3 is Br and R2, R3, R5, X1 are as defined in table B.

Table A-203 provides 200 compounds A-203.001 to A-203.200 of formula (Ia) wherein R1 is CH2O(C═O)CH(CH3)3, X2 is CCF3, X3 is F and R2, R3, R5, X1 are as defined in table B.

Table A-204 provides 200 compounds A-204.001 to A-204.200 of formula (Ia) wherein R1 is CH2O(C═O)CH(CH3)3, X2 is CCF3, X3 is CF₃ and R2, R3, R5, X1 are as defined in table B.

Table A-205 provides 200 compounds A-205.001 to A-205.200 of formula (Ia) wherein R1 is CH2O(C═O)CH(CH3)3, X2 is CCF3, X3 is H and R2, R3, R5, X1 are as defined in table B.

Table A-206 provides 200 compounds A-206.001 to A-206.200 of formula (Ia) wherein R1 is CH2O(C═O)CH(CH3)3, X2 is CH, X3 is Cl and R2, R3, R5, X1 are as defined in table B.

Table A-207 provides 200 compounds A-207.001 to A-207.200 of formula (Ia) wherein R1 is CH2O(C═O)CH(CH3)3, X2 is CH, X3 is Br and R2, R3, R5, X1 are as defined in table B.

Table A-208 provides 200 compounds A-208.001 to A-208.200 of formula (Ia) wherein R1 is CH2O(C═O)CH(CH3)3, X2 is CH, X3 is F and R2, R3, R5, X1 are as defined in table B.

Table A-209 provides 200 compounds A-209.001 to A-209.200 of formula (Ia) wherein R1 is CH2O(C═O)CH(CH3)3, X2 is CH, X3 is CF₃ and R2, R3, R5, X1 are as defined in table B.

Table A-210 provides 200 compounds A-210.001 to A-210.200 of formula (Ia) wherein R1 is CH2O (C═O)CH(CH3)3, X2 is CH, X3 is H and R2, R3, R5, X1 are as defined in table B.

Examples of compounds of formula (Int-1) made available are X^{B} is bromo, chloro, iodo, cyano, formyl, CH═NOH or acetyl, A², A³ and A⁴ are each CH, A¹ is CR⁵, R6 is S(O)₂R2, wherein R1, R2 and R5 are the substituents defined in Table Q below. Each of Tables C-1 to C-7, which follow the Table Q below, make available compounds of the formula (Int-1) in which R2 and R5 are the substituents defined in Table Q and X^{B} is as defined in the relevant Table C-1 to C-7.

TABLE P
Substituent definitions of R¹, R² and R⁵:

| Index | R2 | R5 | R1 |
|---|---|---|---|
| 1 | CH3 | Cl | CH2O(═O)OCH3 |
| 2 | CH3 | Br | CH2O(═O)OCH3 |
| 3 | CH3 | CF3 | CH2O(═O)OCH3 |
| 4 | CH3 | CH3 | CH2O(═O)OCH3 |
| 5 | CH3 | CH2CH3 | CH2O(═O)OCH3 |
| 6 | CH2CH3 | Cl | CH2O(═O)OCH3 |
| 7 | CH2CH3 | Br | CH2O(═O)OCH3 |
| 8 | CH2CH3 | CF3 | CH2O(═O)OCH3 |
| 9 | CH2CH3 | CH3 | CH2O(═O)OCH3 |
| 10 | CH2CH3 | CH2CH3 | CH2O(═O)OCH3 |
| 11 | CH2F | Cl | CH2O(═O)OCH3 |
| 12 | CH2F | Br | CH2O(═O)OCH3 |
| 13 | CH2F | CF3 | CH2O(═O)OCH3 |
| 14 | CH2F | CH3 | CH2O(═O)OCH3 |
| 15 | CH2F | CH2CH3 | CH2O(═O)OCH3 |
| 16 | CHF2 | Cl | CH2O(═O)OCH3 |
| 17 | CHF2 | Br | CH2O(═O)OCH3 |
| 18 | CHF2 | CF3 | CH2O(═O)OCH3 |
| 19 | CHF2 | CH3 | CH2O(═O)OCH3 |
| 20 | CHF2 | CH2CH3 | CH2O(═O)OCH3 |
| 21 | CH3 | Cl | CH2N(CH3) |
| | | | (═O)OCH3 |
| 22 | CH3 | Br | CH2N(CH3) |
| | | | (═O)OCH3 |
| 23 | CH3 | CF3 | CH2N(CH3) |
| | | | (═O)OCH3 |
| 24 | CH3 | CH3 | CH2N(CH3) |
| | | | (═O)OCH3 |
| 25 | CH3 | CH2CH3 | CH2N(CH3) |
| | | | (═O)OCH3 |
| 26 | CH2CH3 | Cl | CH2N(CH3) |
| | | | (═O)OCH3 |
| 27 | CH2CH3 | Br | CH2N(CH3) |
| | | | (═O)OCH3 |
| 28 | CH2CH3 | CF3 | CH2N(CH3) |
| | | | (═O)OCH3 |
| 29 | CH2CH3 | CH3 | CH2N(CH3) |
| | | | (═O)OCH3 |
| 30 | CH2CH3 | CH2CH3 | CH2N(CH3) |
| | | | (═O)OCH3 |
| 31 | CH2F | Cl | CH2N(CH3) |
| | | | (═O)OCH3 |
| 32 | CH2F | Br | CH2N(CH3) |
| | | | (═O)OCH3 |
| 33 | CH2F | CF3 | CH2N(CH3) |
| | | | (═O)OCH3 |
| 34 | CH2F | CH3 | CH2N(CH3) |
| | | | (═O)OCH3 |
| 35 | CH2F | CH2CH3 | CH2N(CH3) |
| | | | (═O)OCH3 |
| 36 | CHF2 | Cl | CH2N(CH3) |
| | | | (═O)OCH3 |
| 37 | CHF2 | Br | CH2N(CH3) |
| | | | (═O)OCH3 |
| 38 | CHF2 | CF3 | CH2N(CH3) |
| | | | (═O)OCH3 |
| 39 | CHF2 | CH3 | CH2N(CH3) |
| | | | (═O)OCH3 |
| 40 | CHF2 | CH2CH3 | CH2N(CH3) |
| | | | (═O)OCH3 |
| 41 | CH3 | Cl | CH2O(═O)OCH2CH3 |
| 42 | CH3 | Br | CH2O(═O)OCH2CH3 |
| 43 | CH3 | CF3 | CH2O(═O)OCH2CH3 |
| 44 | CH3 | CH3 | CH2O(═O)OCH2CH3 |
| 45 | CH3 | CH2CH3 | CH2O(═O)OCH2CH3 |
| 46 | CH2CH3 | Cl | CH2O(═O)OCH2CH3 |
| 47 | CH2CH3 | Br | CH2O(═O)OCH2CH3 |
| 48 | CH2CH3 | CF3 | CH2O(═O)OCH2CH3 |
| 49 | CH2CH3 | CH3 | CH2O(═O)OCH2CH3 |
| 50 | CH2CH3 | CH2CH3 | CH2O(═O)OCH2CH3 |
| 51 | CH2F | Cl | CH2O(═O)OCH2CH3 |
| 52 | CH2F | Br | CH2O(═O)OCH2CH3 |
| 53 | CH2F | CF3 | CH2O(═O)OCH2CH3 |
| 54 | CH2F | CH3 | CH2O(═O)OCH2CH3 |
| 55 | CH2F | CH2CH3 | CH2O(═O)OCH2CH3 |
| 56 | CHF2 | Cl | CH2O(═O)OCH2CH3 |
| 57 | CHF2 | Br | CH2O(═O)OCH2CH3 |
| 58 | CHF2 | CF3 | CH2O(═O)OCH2CH3 |
| 59 | CHF2 | CH3 | CH2O(═O)OCH2CH3 |
| 60 | CHF2 | CH2CH3 | CH2O(═O)OCH2CH3 |
| 61 | CH3 | Cl | CH2O(═O)CH3 |
| 62 | CH3 | Br | CH2O(═O)CH3 |
| 63 | CH3 | CF3 | CH2O(═O)CH3 |
| 64 | CH3 | CH3 | CH2O(═O)CH3 |
| 65 | CH3 | CH2CH3 | CH2O(═O)CH3 |
| 66 | CH2CH3 | Cl | CH2O(═O)CH3 |
| 67 | CH2CH3 | Br | CH2O(═O)CH3 |
| 68 | CH2CH3 | CF3 | CH2O(═O)CH3 |
| 69 | CH2CH3 | CH3 | CH2O(═O)CH3 |
| 70 | CH2CH3 | CH2CH3 | CH2O(═O)CH3 |
| 71 | CH2F | Cl | CH2O(═O)CH3 |
| 72 | CH2F | Br | CH2O(═O)CH3 |
| 73 | CH2F | CF3 | CH2O(═O)CH3 |
| 74 | CH2F | CH3 | CH2O(═O)CH3 |
| 75 | CH2F | CH2CH3 | CH2O(═O)CH3 |
| 76 | CHF2 | Cl | CH2O(═O)CH3 |
| 77 | CHF2 | Br | CH2O(═O)CH3 |
| 78 | CHF2 | CF3 | CH2O(═O)CH3 |
| 79 | CHF2 | CH3 | CH2O(═O)CH3 |
| 80 | CHF2 | CH2CH3 | CH2O(═O)CH3 |
| 81 | CH3 | Cl | CH2O(═O)CH(CH3)2 |
| 82 | CH3 | Br | CH2O(═O)CH(CH3)2 |
| 83 | CH3 | CF3 | CH2O(═O)CH(CH3)2 |
| 84 | CH3 | CH3 | CH2O(═O)CH(CH3)2 |
| 85 | CH3 | CH2CH3 | CH2O(═O)CH(CH3)2 |
| 86 | CH2CH3 | Cl | CH2O(═O)CH(CH3)2 |
| 87 | CH2CH3 | Br | CH2O(═O)CH(CH3)2 |
| 88 | CH2CH3 | CF3 | CH2O(═O)CH(CH3)2 |
| 89 | CH2CH3 | CH3 | CH2O(═O)CH(CH3)2 |
| 90 | CH2CH3 | CH2CH3 | CH2O(═O)CH(CH3)2 |
| 91 | CH2F | Cl | CH2O(═O)CH(CH3)2 |
| 92 | CH2F | Br | CH2O(═O)CH(CH3)2 |
| 93 | CH2F | CF3 | CH2O(═O)CH(CH3)2 |
| 94 | CH2F | CH3 | CH2O(═O)CH(CH3)2 |
| 95 | CH2F | CH2CH3 | CH2O(═O)CH(CH3)2 |
| 96 | CHF2 | Cl | CH2O(═O)CH(CH3)2 |
| 97 | CHF2 | Br | CH2O(═O)CH(CH3)2 |
| 98 | CHF2 | CF3 | CH2O(═O)CH(CH3)2 |
| 99 | CHF2 | CH3 | CH2O(═O)CH(CH3)2 |
| 100 | CHF2 | CH2CH3 | CH2O(═O)CH(CH3)2 |
| 101 | CH3 | Cl | CH2O(═O)CHcPr |
| 102 | CH3 | Br | CH2O(═O)CHcPr |
| 103 | CH3 | CF3 | CH2O(═O)CHcPr |
| 104 | CH3 | CH3 | CH2O(═O)CHcPr |
| 105 | CH3 | CH2CH3 | CH2O(═O)CHcPr |
| 106 | CH2CH3 | Cl | CH2O(═O)CHcPr |
| 107 | CH2CH3 | Br | CH2O(═O)CHcPr |
| 108 | CH2CH3 | CF3 | CH2O(═O)CHcPr |
| 109 | CH2CH3 | CH3 | CH2O(═O)CHcPr |
| 110 | CH2CH3 | CH2CH3 | CH2O(═O)CHcPr |
| 111 | CH2F | Cl | CH2O(═O)CHcPr |
| 112 | CH2F | Br | CH2O(═O)CHcPr |
| 113 | CH2F | CF3 | CH2O(═O)CHcPr |
| 114 | CH2F | CH3 | CH2O(═O)CHcPr |
| 115 | CH2F | CH2CH3 | CH2O(═O)CHcPr |
| 116 | CHF2 | Cl | CH2O(═O)CHcPr |
| 117 | CHF2 | Br | CH2O(═O)CHcPr |
| 118 | CHF2 | CF3 | CH2O(═O)CHcPr |
| 119 | CHF2 | CH3 | CH2O(═O)CHcPr |
| 120 | CHF2 | CH2CH3 | CH2O(═O)CHcPr |
| 121 | CH3 | Cl | CH2O(═O)CH(CH3)3 |
| 122 | CH3 | Br | CH2O(═O)CH(CH3)3 |
| 123 | CH3 | CF3 | CH2O(═O)CH(CH3)3 |
| 124 | CH3 | CH3 | CH2O(═O)CH(CH3)3 |
| 125 | CH3 | CH2CH3 | CH2O(═O)CH(CH3)3 |
| 126 | CH2CH3 | Cl | CH2O(═O)CH(CH3)3 |
| 127 | CH2CH3 | Br | CH2O(═O)CH(CH3)3 |
| 128 | CH2CH3 | CF3 | CH2O(═O)CH(CH3)3 |
| 129 | CH2CH3 | CH3 | CH2O(═O)CH(CH3)3 |
| 130 | CH2CH3 | CH2CH3 | CH2O(═O)CH(CH3)3 |
| 131 | CH2F | Cl | CH2O(═O)CH(CH3)3 |
| 132 | CH2F | Br | CH2O(═O)CH(CH3)3 |
| 133 | CH2F | CF3 | CH2O(═O)CH(CH3)3 |
| 134 | CH2F | CH3 | CH2O(═O)CH(CH3)3 |
| 135 | CH2F | CH2CH3 | CH2O(═O)CH(CH3)3 |
| 136 | CHF2 | Cl | CH2O(═O)CH(CH3)3 |
| 137 | CHF2 | Br | CH2O(═O)CH(CH3)3 |
| 138 | CHF2 | CF3 | CH2O(═O)CH(CH3)3 |
| 139 | CHF2 | CH3 | CH2O(═O)CH(CH3)3 |
| 140 | CHF2 | CH2CH3 | CH2O(═O)CH(CH3)3 |

Table CC-1 provides 140 compounds C-1.01 to C-1.140 of formula (Int-1) wherein X^{B} is bromo and R² and R⁵ are as defined in Table Q.

Table C-2 provides 140 compounds C-2.01 to C-2.140 of formula (Int-1) wherein X^{B} is chloro and R² and R⁵ are as defined in Table Q.

Table C-3 provides 140 compounds C-3.01 to C-3.140 of formula (Int-1) wherein X^{B} is iodo and R² and R⁵ are as defined in Table Q.

Table C-4 provides 140 compounds C-4.01 to C-4.140 of formula (Int-1) wherein X^{B} is cyano and R² and R⁵ are as defined in Table Q.

Table C-5 provides 140 compounds C-5.01 to C-5.140 of formula (Int-1) wherein X^{B} is formyl and R² and R⁵ are as defined in Table Q.

Table C-6 provides 140 compounds C-6.01 to C-6.140 of formula (Int-1) wherein X^{B} is CH═NOH and R² and R⁵ are as defined in Table Q.

Table C-7 provides 140 compounds C-7.01 to C-7.140 of formula (Int-1) wherein X^{B} is acetyl and R² and R⁵ are as defined in Table Q.

Examples of compounds of formula (Int-II) made available are those where X^{c} is CH₂C₁, CH₂Br, CH═C(CF₃)(3-chloro-5-trifluoromethyl-phenyl), CH═C(CF₃)(3-bromo-5-trifluoromethyl-phenyl), CH═C(CF₃)(3,5-dichloro-4-fluoro-phenyl), CH═C(CF₃)(3,4,5-trichloro-phenyl), CH₂C(OH)(CF₃)(3-chloro-5-trifluoromethyl-phenyl), CH₂C(OH)(CF₃)(3-bromo-5-trifluoromethyl-phenyl), CH₂C(OH)(CF₃)(3,5-dichloro-4-fluoro-phenyl) or CH₂C(OH)(CF₃)(3,4,5-trichloro-phenyl), A², A³ and A⁴ are each CH, A¹ is CR⁵ and R⁶ is S(O)₂R², wherein R¹, R² and R⁵ correspond to the substituents R¹, R² and R⁵ as defined in Table Q above. Each of Tables D-1 to D-10, which follow below, make available compounds of the formula (Int-II) in which R¹, R² and R⁵ are the substituents defined in Table Q and X^{C} is as defined in the relevant Table D-1 to D-10.

Table D-1 provides 140 compounds D-1.01 to D-1.140 of formula (Int-II) wherein X^{C} is CH₂Cl and R² and R⁵ are as defined in Table Q.

Table D-2 provides 140 compounds D-2.01 to D-2.140 of formula (Int-II) wherein X^{C} is CH₂Br and R² and R⁵ are as defined in Table Q.

Table D-3 provides 140 compounds D-3.01 to D-3.140 of formula (Int-II) wherein X^{C} is CH═C(CF₃)(3-chloro-5-trifluoromethyl-phenyl) and R² and R⁵ are as defined in Table Q.

Table D-4 provides 140 compounds D-4.01 to D-4.140 of formula (Int-II) wherein X^{C} is CH═C(CF₃)(3-bromo-5-trifluoromethyl-phenyl) and R² and R⁵ are as defined in Table Q.

Table D-5 provides 140 compounds D-5.01 to D-5.140 of formula (Int-II) wherein X^{C} is CH═C(CF₃)(3,5-dichloro-4-fluoro-phenyl) and R² and R⁵ are as defined in Table Q.

Table D-6 provides 140 compounds D-6.01 to D-6.140 of formula (Int-II) wherein X^{C} is CH═C(CF₃)(3,4,5-trichloro-phenyl) and R² and R⁵ are as defined in Table Q.

Table D-7 provides 140 compounds D-7.01 to D-7.140 of formula (Int-II) wherein X^{C} is CH₂C(OH)(CF₃)(3-chloro-5-trifluoromethyl-phenyl) and R² and R⁵ are as defined in Table Q.

Table D-8 provides 140 compounds D-8.01 to D-8.140 of formula (Int-II) wherein X^{C} is CH₂C(OH)(CF₃)(3-bromo-5-trifluoromethyl-phenyl) and R² and R⁵ are as defined in Table Q.

Table D-9 provides 140 compounds D-9.01 to D-9.140 of formula (Int-II) wherein X^{C} is CH₂C(OH)(CF₃)(3,5-dichloro-4-fluoro-phenyl) and R² and R⁵ are as defined in Table Q.

Table D-10 provides 140 compounds D-10.01 to D-10.140 of formula (Int-II) wherein X^{C} is CH₂C(OH)(CF₃)(3,4,5-trichloro-phenyl) and R² and R⁵ are as defined in Table Q.

The compounds of formula (I) according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate.

Examples of the abovementioned animal pests are:

from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,

### Haematopinus spp., Linognathus spp., Pediculus spp., Pemphigus spp. and Phylloxera spp.;

from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypi-ela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,

### Damalinea spp. and Trichodectes spp.;

from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,

### Liposcelis spp.;

from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca(preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

Crops of useful plants in which the composition according to the invention can be used include perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include 6-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents. A further object of the invention is therefore a substrate selected from nonwoven and fabric material comprising a composition which contains a compound of formula (I).

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO2008/151984, WO2003/034823, U.S. Pat. No. 5,631,072, WO2005/64072, WO 2006/128870, EP 1724392, WO2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticoillis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. aponica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:
Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattelagermanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.
Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirexjuvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 l/ha, especially from 10 to 1000 l/ha.

Preferred formulations can have the following compositions (weight %):

| | |
|---|---|
| Emulsifiable concentrates: | |
| active ingredient: | 1 to 95%, preferably 60 to 90% |
| surface-active agent: | 1 to 30%, preferably 5 to 20% |
| liquid carrier: | 1 to 80%, preferably 1 to 35% |
| Dusts: | |
| active ingredient: | 0.1 to 10%, preferably 0.1 to 5% |
| solid carrier: | 99.9 to 90%, preferably 99.9 to 99% |
| Suspension concentrates: | |
| active ingredient: | 5 to 75%, preferably 10 to 50% |
| water: | 94 to 24%, preferably 88 to 30% |
| surface-active agent: | 1 to 40%, preferably 2 to 30% |
| Wettable powders: | |
| active ingredient: | 0.5 to 90%, preferably 1 to 80% |
| surface-active agent: | 0.5 to 20%, preferably 1 to 15% |
| solid carrier: | 5 to 95%, preferably 15 to 90% |
| Granules: | |
| active ingredient: | 0.1 to 30%, preferably 0.1 to 15% |
| solid carrier: | 99.5 to 70%, preferably 97 to 85% |

The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether | 4% |
| (35 mol of ethylene oxide) | |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| active ingredients | 15% |
| sodium lignosulfonate | 2% |
| carboxymethylcellu lose | 1% |
| Kaolin | 82% |

The combination is mixed and ground with the adjuvants, and the mixture is moistened with water. The mixture is extruded and then dried in a stream of air.

| Coated granules | |
|---|---|
| active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

| Suspension concentrate | |
|---|---|
| active ingredients | 40% |
| propylene glycol | 10% |
| nonylphenol polyethylene glycol | 6% |
| ether (15 mol of ethylene oxide) | |
| Sodium lignosulfonate | 10% |
| carboxymethylcellulose | 1% |
| silicone oil (in the form of a | 1% |
| 75% emulsion in water) | |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

| Flowable concentrate for seed treatment | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form | 0.5% |
| of a 20% solution in water) | |
| monoazo-pigment calcium salt | 5% |
| Silicone oil (in the form of a 75% | 0.2% |
| emulsion in water) | |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use. Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula (I) with active ingredients are preferred (the abbreviation “TX” means “one compound selected from the group consisting of the compounds described in Table A-1 to A-100 and A of the present invention”):

an adjuvant selected from the group of substances consisting of petroleum oils (alternative name) (628)+TX,
an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos (alternative name) [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate (alternative name)+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben (alternative name)+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin 1(696)+TX, cinerin 11 (696)+TX, cinerins (696)+TX, clofentezine (158)+TX, closantel (alternative name) [CCN]+TX, coumaphos (174)+TX, crotamiton (alternative name) [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S(1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos (alternative name)+TX, dicofol (242)+TX, dicrotophos (243)+TX, dienochlor (1071)+TX, dimefox (1081)+TX, dimethoate (262)+TX, dinactin (alternative name) (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram (alternative name) [CCN]+TX, disulfoton (278)+TX, DNOC (282)+TX, dofenapyn (1113)+TX, doramectin (alternative name) [CCN]+TX, endosulfan (294)+TX, endothion (1121)+TX, EPN (297)+TX, eprinomectin (alternative name) [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad (alternative name)+TX, fen-pyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (alternative name) (473)+TX, isopropyl O-(methoxyaminothiophosphoryl)salicylate (IUPAC name) (473)+TX, ivermectin (alternative name) [CCN]+TX, jasmolin 1 (696)+TX, jasmolin 11 (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen (alternative name) [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime (alternative name) [CCN]+TX, mipafox (1293)+TX, monocrotophos (561)+TX, morphothion (1300)+TX, moxidectin (alternative name) [CCN]+TX, naled (567)+TX, NC-184 (compound code)+TX, NC-512 (compound code)+TX, nifluridide (1309)+TX, nikkomycins (alternative name) [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (alternative name) (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (alternative name) (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin 1 (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos (alternative name)+TX, selamectin (alternative name) [CCN]+TX, SI-0009 (compound code)+TX, sophamide (1402)+TX, spirodiclofen (738)+TX, spiromesifen (739)+TX, SSI-121 (development code) (1404)+TX, sulfiram (alternative name) [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam (alternative name)+TX, tetrachlorvinphos (777)+TX, tetradifon (786)+TX, tetranactin (alternative name) (653)+TX, tetrasul (1425)+TX, thiafenox (alternative name)+TX, thiocarboxime (1431)+TX, thiofanox (800)+TX, thiometon (801)+TX, thioquinox (1436)+TX, thuringiensin (alternative name) [CCN]+TX, triamiphos (1441)+TX, triarathene (1443)+TX, triazophos (820)+TX, triazuron (alternative name)+TX, trichlorfon (824)+TX, trifenofos (1455)+TX, trinactin (alternative name) (653)+TX, vamidothion (847)+TX, vaniliprole [CCN] and YI-5302 (compound code)+TX,
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX, an anthelmintic selected from the group of substances consisting of abamectin (1)+TX, crufomate (1011)+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX,
an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX,
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX,
a soil sterilant selected from the group of substances consisting of iodomethane (IUPAC name) (542) and methyl bromide (537)+TX,
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX,
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B (alternative name) (839)+TX, trimedlure B₂ (alternative name) (839)+TX, trimedlure C (alternative name) (839) and trunc-call (alternative name) [CCN]+TX,
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX, an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion (alternative name) [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin (alternative name) [CCN]+TX, allyxycarb (866)+TX, alpha-cypermethrin (202)+TX, alpha-ecdysone (alternative name) [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (alternative name) (41)+TX, azamethiphos (42)+TX, azinphos-ethyl (44)+TX, azinphos-methyl (45)+TX, azothoate (889)+TX, Bacillus thuringiensis delta endotoxins (alternative name) (52)+TX, barium hexafluorosilicate (alternative name) [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (alternative name) (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate (alternative name)+TX, bromfenvinfos (914)+TX, bromocyclen (918)+TX, bromo-DDT (alternative name) [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben (alternative name)+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (alternative name) (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, CDCl3 [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin 1 (696)+TX, cinerin II (696)+TX, cinerins (696)+TX, cis-resmethrin (alternative name)+TX, cismethrin (80)+TX, clocythrin (alternative name)+TX, cloethocarb (999)+TX, closantel (alternative name) [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton (alternative name) [CCN]+TX, crotoxyphos (1010)+TX, crufomate (1011)+TX, cryolite (alternative name) (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate (alternative name) [CCN]+TX, d-limonene (alternative name) [CCN]+TX, d-tetramethrin (alternative name) (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos (alternative name)+TX, dicresyl (alternative name) [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor (alternative name) [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin (alternative name) [CCN]+TX, DSP (1115)+TX, ecdysterone (alternative name) [CCN]+TX, El 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin (alternative name) [CCN]+TX, esfenvalerate (302)+TX, etaphos (alternative name) [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (alternative name) (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad (alternative name)+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (alternative name) (473)+TX, isodrin (1235)+TX, isofenphos (1236)+TX, isolane (1237)+TX, isoprocarb (472)+TX, isopropyl O-(methoxy-aminothiophosphoryl)salicylate (IUPAC name) (473)+TX, isoprothiolane (474)+TX, isothioate (1244)+TX, isoxathion (480)+TX, ivermectin (alternative name) [CCN]+TX, jasmolin I (696)+TX, jasmolin II (696)+TX, jodfenphos (1248)+TX, juvenile hormone I (alternative name) [CCN]+TX, juvenile hormone II (alternative name) [CCN]+TX, juvenile hormone III (alternative name) [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (alternative name) (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (alternative name) (533)+TX, methoxychlor (534)+TX, methoxyfenozide (535)+TX, methyl bromide (537)+TX, methyl isothiocyanate (543)+TX, methylchloroform (alternative name) [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime (alternative name) [CCN]+TX, mipafox (1293)+TX, mirex (1294)+TX, monocrotophos (561)+TX, morphothion (1300)+TX, moxidectin (alternative name) [CCN]+TX, naftalofos (alternative name) [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron (alternative name) [CCN]+TX, pentachlorophenol (623)+TX, pentachlorophenyl laurate (IUPAC name) (623)+TX, permethrin (626)+TX, petroleum oils (alternative name) (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I (alternative name) [CCN]+TX, precocene II (alternative name) [CCN]+TX, precocene III (alternative name) [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia (alternative name) [CCN]+TX, quinalphos (711)+TX, quinalphos-methyl (1376)+TX, quinothion (1380)+TX, quintiofos (1381)+TX, R-1492 (development code) (1382)+TX, rafoxanide (alternative name) [CCN]+TX, resmethrin (719)+TX, rotenone (722)+TX, RU 15525 (development code) (723)+TX, RU 25475 (development code) (1386)+TX, ryania (alternative name) (1387)+TX, ryanodine (traditional name) (1387)+TX, sabadilla (alternative name) (725)+TX, schradan (1389)+TX, sebufos (alternative name)+TX, selamectin (alternative name) [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (alternative name) (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam (alternative name)+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox (alternative name)+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin (alternative name) [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron (alternative name)+TX, trichlorfon (824)+TX, trichlormetaphos-3 (alternative name) [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (alternative name) (725)+TX, veratrine (alternative name) (725)+TX, XMC (853)+TX, xylylcarb (854)+TX, YI-5302 (compound code)+TX, zeta-cypermethrin (205)+TX, zetamethrin (alternative name)+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO2005/077934)+TX,
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX,
a nitrification inhibitor selected from the group of substances consisting of potassium ethylxanthate [CCN] and nitrapyrin (580)+TX,
a plant activator selected from the group of substances consisting of acibenzolar (6)+TX, acibenzolar-S-methyl (6)+TX, probenazole (658) and Reynoutria sachalinensis extract (alternative name) (720)+TX,
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,
a virucide selected from the group of substances consisting of imanin (alternative name) [CCN] and ribavirin (alternative name) [CCN]+TX,
a wound protectant selected from the group of substances consisting of mercuric oxide (512)+TX, octhilinone (590) and thiophanate-methyl (802)+TX,
and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebucon-azole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzolar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimetho-morph [110488-70-5]+TX, SYP-LI90 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (dislosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX,1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX; lancotrione [1486617-21-3]+TX, florpyrauxifen [943832-81-3]]+TX, ipfentrifluconazole[1417782-08-1]+TX, mefentrifluconazole [1417782-03-6]+TX, quinofumelin [861647-84-9]+TX, chloroprallethrin [399572-87-3]+TX, cyhalodiamide [1262605-53-7]]+TX, fluazaindolizine [1254304-22-7]+TX, fluxametamide [928783-29-3]+TX, epsilon-metofluthrin [240494-71-7]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1]+TX, kappa-tefluthrin [391634-71-2]+TX, fenpicoxamid [517875-34-2]+TX; fluindapyr [1383809-87-7]+TX; alpha-bromadiolone [28772-56-7]+TX; flupyrimin [1689566-03-7]+TX; benzpyrimoxan [1449021-97-9]+TX; acynonapyr [1332838-17-1]+TX; inpyrfluxam [1352994-67-2]+TX, isoflucypram [1255734-28-1]+TX; rescalure [64309-03-1]+TX; aminopyrifen [1531626-08-0]+TX; tyclopyrazoflor [1477919-27-9]+TX; and spiropidion [1229023-00-0]+TX; and microbials including: Acinetobacterlwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis CrylAb+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone—formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera)+TX, Virgibaclillus marismortui+TX, Xanthomonas campestris pv. Poae (Camperico®)+TX, Xenorhabdus bovienii+TX, Xenorhabdus nematophilus; and Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol)+TX, Chenopodium ambrosioides near ambrosioides (Requiem®)+TX, Chrysanthemum extract (Crisant)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®); and
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline swirskii®+TX, Swirskii-Mite®)+TX, Amblyseius womersleyi (WomerMite)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline Hm®+TX, Larvanem-M®)+TX, Hippodamia convergens+TX, Hypoaspis aculeifer (Aculeifer-System®+TX, Entomite-A)+TX, Hypoaspis miles (Hypoline M®+TX, Entomite-M)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline i)+TX, Orius laevigatus (Thripor-L®+TX, Oriline I®)+TX, Orius majusculus (Oriline M®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline Sf®+TX, Scia-Rid®+TX, Entonem®)+TX, Steinernema kraussei (Nemasys L®+TX, BioNem L®+TX, Exhibitline Srb®)+TX, Steinernema riobrave (BioVector®+TX, BioVektor)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; and other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®+TX, Ni-HIBIT Gold CST®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula (I) selected from Table A-1 to A-100 and A with active ingredients described above comprises a compound selected from Table A-1 to A-100 and A and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from Table A-1 to A-100 and A and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Table A-1 to A-100 and A and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula (I). The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### PREPARATORY EXAMPLES

The following Examples illustrate, but do not limit, the invention.

The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm, 0.4 ppm, 0.2 ppm, 0.1 ppm or even at lower concentrations.

“Mp” means melting point in ° C. Free radicals represent methyl groups. ¹H and ¹⁹F NMR measurements were recorded on Brucker 400 MHz or 300 MHz spectrometers, chemical shifts are given in ppm relative to a TMS standard. Spectra measured in deuterated solvents as indicated (br means that the following signal pattern is broad e.g. br s is a broad singlet; s=singlet; d=doublet; m=multiplet).

### Example 1: Preparation of [[4-[(5S)-5-(3,5-dichloro-4-fluoro-phenyl)-5-(trifluoromethyl)-4H-isoxazol-3-yl]-2-methyl-benzoyl]-[1-(difluoromethylsulfonyl)azetidin-3-yl]amino]methyl methyl carbonate (Compound A2)

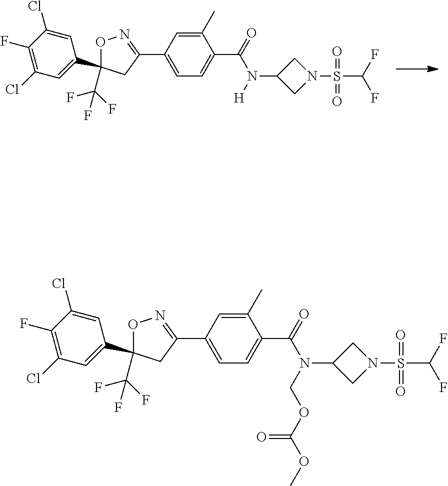 [SMILES: Cc1cc(C2=NO[C@@](c3cc(Cl)c(F)c(Cl)c3)(C(F)(F)F)C2)ccc1C(=O)NC1CN(S(=O)(=O)C(F)F)C1 ; COC(=O)OCN(C(=O)c1ccc(C2=NO[C@@](c3cc(Cl)c(F)c(Cl)c3)(C(F)(F)F)C2)cc1C)C1CN(S(=O)(=O)C(F)F)C1]

To a solution of 4-[(5S)-5-(3,5-dichloro-4-fluoro-phenyl)-5-(trifluoromethyl)-4H-isoxazol-3-yl]-N-[1-(difluoromethylsulfonyl)azetidin-3-yl]-2-methyl-benzamide (1000 mg) in tetrahydrofuran (8.23 mL) was added chloromethyl methylcarbonate (1.062 g) at −40° C., under argonatmosphere. The reaction mixture was stirred for 30 min and then a potassium tert-butoxide solution in tetrahydrofuran (2.48 mL, 1M) was added at −40° C. and the mixture was stirred for 90 min, then it was warmed to room temperature and stirred for 4 hours. To the obtained mixture was added water and the water phase was separated and extracted with ethyl acetate. The organic phases were combined, dried over sodium sulfate, filtered and evaporated under reduced pressure. The obtained crude was purified by chromatography over silica gel with ethyl acetate-cyclohexane mixture as an eluent (gradient from 0:1 to 1:1), then a reverse phase chromatography to give [[4-[(5S)-5-(3,5-dichloro-4-fluoro-phenyl)-5-(trifluoromethyl)-4H-isoxazol-3-yl]-2-methyl-benzoyl]-[1-(difluoromethylsulfonyl)azetidin-3-yl]amino]methyl methyl carbonate (38 mg) as foam. 1H NMR (400 MHz, CHLOROFORM-d) δ ppm 7.53-7.64 (m, 4H) 7.25-7.32 (m, 1H) 6.04-6.44 (m, 1H) 5.24 (m, 2H) 4.92 (m, 1H) 4.38-4.59 (m, 4H) 4.05-4.18 (m, 1H) 3.78 (s, 3H) 3.69 (d, 1H) 2.35 (s, 3H). 19F NMR (377 MHz, CHLOROFORM-d) δ ppm−79.46 (s, 3F)−113.44 (s, 1F)−121.09 (s, 2F).

The other compounds were prepared in a similar manner as Example 1.

TABLE A
Compounds of formula AA
(AA)

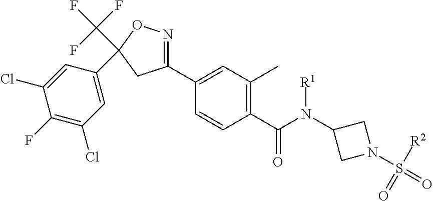

| Compound | | |
|---|---|---|
| number | R1 | R2 |
| A1 | —CH2N(CH3)C(O)OCH3 | CHF2 |
| A2 | —CH2OC(O)OCH3 | CHF2 |

TABLE A-DATA
NMR data of Compounds of formula AA as described in Table A:

| Com- | | |
|---|---|---|
| pound | | |
| Number | 1H NMR | 19F NMR |
| A1 | 1H NMR (400 MHz, CDCl3) δ ppm | 19F NMR (376 MHz, |
| | 7.55-7.63 (m, 4 H) 7.29-7.41 (m, 1 H) | CDCl3) δ ppm-79.53 |
| | 6.03-6.45 (m, 1 H) 4.27-5.00 (m, 6 H) | (s, 3 F)-113.38 (s, 1 F)- |
| | 4.01-4.16 (m, 1 H) 3.66-3.81 (m, 4 H) | 121.07 (s, 2 F) |
| | 2.51-3.20 (m, 3 H) 2.39 (br s, 3 H) | |
| A2 | 1H NMR (400 MHz, CDCl3) δ ppm | 19F NMR (376 MHz, |
| | 7.53-7.64 (m, 4 H) 7.25-7.32 (m, 1 H) | CDCl3) δ ppm-79.46 |
| | 6.04-6.44 (m, 1 H) 5.24 (m, 2 H) 4.92 | (s, 3 F)-113.44 (s, 1 F)- |
| | (m, 1 H) 4.38-4.59 (m, 4 H) 4.05-4.18 | 121.09 (s, 2 F) |
| | (m, 1 H) 3.78 (s, 3 H) 3.69 (d, 1 H) | |
| | 2.35 (s, 3 H) | |

### Biological Examples

These Examples illustrate the pesticidal/insecticidal properties of compounds of formula (Ia).

Tests were performed as follows:

Euschistus heros(Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: A1, A2

Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: A1, A2

1. A compound of formula (I), 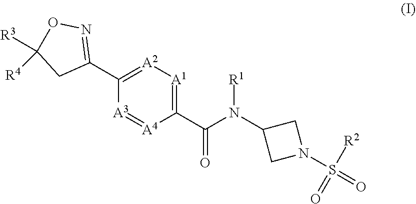
wherein
A¹, A², A³ and A⁴, independently of one another, are C—H, C—R⁵ or nitrogen; wherein not more than two of A¹, A², A³ and A⁴ are N;
R¹ is selected from C₃-C₆cycloalkylcarbonyl, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}) and —R^{1a}C(═O)N(R^{1b})(R^{1c});
R^{1a} is —(CR^{1d}R^{1e})ₙ—;
R^{1b} and R^{1c} are independently selected from H and C₁-C₈alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R² is C₁-C₈alkyl or C₁-C₈haloakyl;
R³ is C₁-C₈haloalkyl;
R⁴ is aryl, aryl substituted by one to three R⁷, heteroaryl or heteroaryl substituted by one to three R⁷;
R⁵ is independently selected from halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy and C₁-C₈alkoxycarbonyl-, or two R⁵ on adjacent carbon atoms together form a —CH₂—CH₂—CH₂— bridge, a —CH═CH—CH═CH— bridge or a —N═CH—CH═CH— bridge; and
R⁷ is independently selected from halogen, cyano, nitro, C₁-C₈alkyl, C₁-C₈haloalkyl, C₁-C₈alkoxy and C₁-C₈haloalkoxy;
or an agrochemically acceptable salt, stereoisomer, enantiomer, tautomer and N-oxide thereof.

2. The compound according to claim 1,
wherein
A¹, A², A³ and A⁴ are independently selected from C—H and C—R⁵;
R¹ is selected from C₃-C₆cycloalkylcarbonyl, —R^{1a}OC(═O)R^{1b}, —R^{1a}OC(═O)OR^{1b},
R^{1a}N(R^{1c})C(═O)OR^{1b}, —R^{1a}OC(═O)NHR^{1b}, —R^{1a}OC(═O)N(R^{1b})(R^{1c}), —R^{1a}C(═O)NHR^{1b}, —and R^{1a}C(═O)N(R^{1b})(R^{1c});
R^{1a} is —(CR^{1d}R^{1e})ₙ—;
R^{1b} and R^{1c} are independently selected from H and C₁-C₈alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R² is C₁-C₈alkyl or C₁-C₈haloakyl;
R³ is C₁-C₈haloalkyl;
R⁴ is phenyl which is unsubstituted or substituted by one to three R⁷;
R⁵ is independently selected from halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy and C₁-C₈alkoxycarbonyl-, or two R⁵ on adjacent carbon atoms together form a —CH₂—CH₂—CH₂— bridge, a —CH═CH—CH═CH— bridge or a —N═CH—CH═CH— bridge; and
R⁷ is independently selected from halogen and C₁-C₈haloalkyl;
or an agrochemically acceptable salt, stereoisomer, enantiomer, tautomer and N-oxide thereof.

3. The compound according to claim 1, wherein
A², A³ and A⁴ are C—H and A¹ is C—R⁵;
R⁵ is C₁-C₄alkyl.

4. The compound according to claim 1, wherein R⁴ is phenyl which is substituted by one to three R⁷ and wherein R⁷ is halogen or C₁-C₈haloalkyl.

5. The compound according to claim 1, wherein R⁴ is selected from 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3,5-dichloro-4-fluoro-phenyl and 3,4,5-trichloro-phenyl.

6. The compound according to claim 1, wherein R³ is —CF₃.

7. The compound according to claim 1, wherein
R² is selected from methyl, ethyl, propyl, isopropyl, CH₂F, CH₂Cl, CH₂Br, CHF₂, CF₃, CH₂CH₂CF₃ and CH₂CF₃.

8. The compound according to claim 1, wherein
R¹ is selected from —R^{1a}OC(═O)OR^{1b} and —R^{1a}N(R^{c})C(═O)OR^{1b}; wherein
R^{1a} is —(CR^{1d}R^{1e})ₙ—;
R^{1b} and R^{1c} are independently selected from C₁-C₈alkyl, wherein each alkyl group is unsubstituted or substituted with one to three halogen atoms or with a cyano group;
R^{1d} and R^{1e} are independently selected from H and C₁-C₄alkyl;
n is 1 or 2.

9. A compound of formula (Int-I) 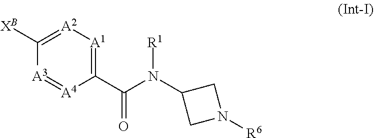
wherein
A¹, A², A³ and A⁴, independently of one another, are C—H, C—R⁵ or nitrogen; wherein not more than two of A¹, A², A³ and A⁴ are N:
R¹ is selected from C₃-C₆cycloalkylcarbonyl, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}) and —R^{1a}C(═O)N(R^{1b})(R^{1c});
R^{1a} is —(CR^{1d}R^{1e})ₙ—;
R^{1b} and R^{1c} are independently selected from H and C₁-C₈alkyl, wherein each alkyl group is unsubstituted or substituted with one to three halogen atoms or with a cyano group;
R^{1d} and R^{1e} are independently selected from H and C₁-C₄alkyl:
n is selected from 1, 2, 3 and 4;
R² is C₁-C₈alkyl or C₁-C₈haloakyl;
R⁵ is independently selected from halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy and C₁-C₈alkoxycarbonyl-, or two R⁵ on adjacent carbon atoms together form a —CH₂—CH₂—CH₂— bridge, a —CH═CH—CH═CH— bridge or a —N═CH—CH═CH— bridge;
and R⁶ is hydrogen or S(O)₂R² and X^{B} is a halogen, or X^{B} is cyano, formyl, CH═N—OH or acetyl; or a salt or N-oxide thereof.

10. A compound of formula (Int-II) 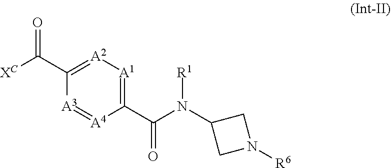
wherein
A¹, A², A³ and A⁴, independently of one another, are C—H, C—R⁵ or nitrogen, wherein not more than two of A¹, A², A³ and A⁴ are N;
R¹ is selected from C₃-C₆cycloalkylcarbonyl, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}) and —R^{1a}C(═O)N(R^{1b})(R^{1c});
R^{1a} is —(CR^{1d}R^{1e})ₙ—;
R^{1b} and R^{1c} are independently selected from H and C₁-C₈alkyl, wherein each alkyl group is unsubstituted or substituted with one to three halogen atoms or with a cyano group:
R^{1d} and R^{1e} are independently selected from H and C₁-C₄alkyl;
n is selected from 1, 2, 3 and 4;
R² is C₁-C₈alkyl or C₁-C₈haloakyl;
R⁵ is independently selected from halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy and C₁-C₈alkoxycarbonyl-, or two R⁵ on adjacent carbon atoms together form a —CH₂—CH₂—CH₂— bridge, a —CH═CH—CH═CH— bridge or a —N═CH—CH═CH— bridge;
R⁶ is hydrogen or S(O)₂R² and X^{C} is CH₂-halogen, wherein halogen is bromo or chloro, CH═C(R³)R⁴ or CH₂C(OH)(R)R⁴;
R³ is C₁-C₈haloalkyl;
R⁴ is aryl, aryl substituted by one to three R⁷, heteroaryl or heteroaryl substituted by one to three R⁷;
R⁷ is independently selected from halogen, cyano, nitro, C₁-C₈alkyl, C₁-C₈haloalkyl, C₁-C₈alkoxy and C₁-C₈haloalkoxy;
or a salt or N-oxide thereof.

11. A compound of formula (Int-III) 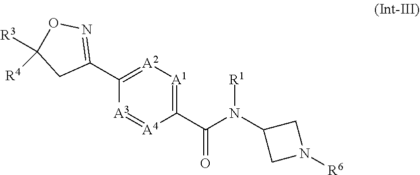
wherein
A¹, A², A³ and A⁴, independently of one another, are C—H, C—R⁵ or nitrogen, wherein not more than two of A¹, A², A³ and A⁴ are N;
R¹ is selected from C₃-C₆cycloalkylcarbonyl, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}) and —R^{1a}C(═O)N(R^{1b})(R^{1c});
R^{1a} is —(CR^{1d}R^{1e})ₙ—;
R^{1b} and R^{1c} are independently selected from H and C₁-C₈alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R³ is C₁-C₈haloalkyl;
R⁴ is aryl, aryl substituted by one to three R⁷, heteroaryl or heteroaryl substituted by one to three R⁷;
R⁵ is independently selected from halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy and C₁-C₈alkoxycarbonyl-, or two R⁵ on adjacent carbon atoms together form a —CH₂—CH₂—CH₂— bridge, a —CH═CH—CH═CH— bridge or a —N═CH—CH═CH— bridge and R⁷ is independently selected from halogen, cyano, nitro, C₁-C₈alkyl, C₁-C₈haloalkyl, C₁-C₈alkoxy and C₁-C₈haloalkoxy;
and R⁶ is hydrogen; or a salt or N-oxide thereof.

12. A pesticidal composition, which comprises at least one compound of formula (I) according to claim 1 or, where appropriate, a tautomer thereof, in each case in free form or in agrochemically utilizable salt form, as active ingredient and at least one auxiliary.

13. A method for controlling pests, which comprises applying a composition according to claim 12 to the pests or their environment with the exception of a method for treatment of the human or animal body by surgery or therapy and diagnostic methods practised on the human or animal body.

14. A method for the protection of seeds from the attack by pests, which comprises treating the seeds or the site, where the seeds are planted, with a composition according to claim 12.

15. The compound according to claim 3 wherein R⁵ is methyl.
